(19) United States
(12) Patent Application Publication
(10) Pub. No.: US 2021/0186081 A1
(43) Pub. Date: Jun. 24, 2021

(54) POUCHED ORAL PRODUCT WITH CANNABINOID

(71) Applicant: NICOVENTURES TRADING LIMITED, London (GB)

(72) Inventors: Anthony Richard Gerardi, Winston-Salem, NC (US); Thomas H. Poole, Winston-Salem, NC (US); Steven Lee Alderman, Lewisville, NC (US)

(21) Appl. No.: 17/187,981

(22) Filed: Mar. 1, 2021

Related U.S. Application Data
(63) Continuation of application No. PCT/IB2020/061346, filed on Dec. 2, 2020.
(60) Provisional application No. 62/945,471, filed on Dec. 9, 2019.

(51) Int. Cl. A24B 13/00; A24B 15/16; A61K 9/00; A61K 31/05; A61K 31/352; A61K 47/38

(52) U.S. Cl. CPC A24B 13/00; A24B 15/16; A61K 9/009; A61K 47/38; A61K 31/05; A61K 31/352; A61K 9/0053

(57) ABSTRACT

The disclosure provides a pouched oral product, including a saliva permeable pouch and an oral product incorporated within the pouch, wherein the pouch material contains a cannabinoid or has a cannabinoid on its outer surface. Further provided is a process for preparing such a pouched oral product. The process includes providing the oral product, incorporating the oral product within the pouch to provide a pouched oral product; and applying a cannabinoid to the surface of the pouch or applying a cannabinoid to the oral product through the pouch.

### CROSS-REFERENCE TO RELATED APPLICATIONS

This application is a continuation of International Application No. PCT/IB2020/061346, filed Dec. 2, 2020, and claims priority to U.S. Provisional Application No. 62/945,471, filed on Dec. 9, 2019, which are incorporated herein by reference in their entirety and for all purposes.

### FIELD

The present disclosure relates to process for preparing a pouched oral product, as well as the pouched oral product itself and packages containing said pouched oral product. In particular, the present disclosure relates to a product, and a process for preparing products, intended for human use. The products are configured for oral use and deliver an active ingredient during use. Such products include a cannabinoid or a product derived from a cannabinoid.

### BACKGROUND

Tobacco may be enjoyed in a so-called “smokeless” form. Particularly popular smokeless tobacco products are employed by inserting some form of processed tobacco or tobacco-containing formulation into the mouth of the user. Conventional formats for such smokeless tobacco products include moist snuff, snus, and chewing tobacco, which are typically formed almost entirely of particulate, granular, or shredded tobacco, and which are either portioned by the user or presented to the user in individual portions, such as in single-use pouches or sachets. Other traditional forms of smokeless products include compressed or agglomerated forms, such as plugs, tablets, or pellets. Alternative product formats, such as tobacco-containing gums and mixtures of tobacco with other plant materials, are also known. See for example, the types of smokeless tobacco formulations, ingredients, and processing methodologies set forth in U.S. Pat. No. 1,376,586 to Schwartz; U.S. Pat. No. 4,513,756 to Pittman et al.; U.S. Pat. No. 4,528,993 to Sensabaugh, Jr. et al.; U.S. Pat. No. 4,624,269 to Story et al.; U.S. Pat. No. 4,991,599 to Tibbetts; U.S. Pat. No. 4,987,907 to Townsend; U.S. Pat. No. 5,092,352 to Sprinkle, III et al.; U.S. Pat. No. 5,387,416 to White et al.; U.S. Pat. No. 6,668,839 to Williams; U.S. Pat. No. 6,834,654 to Williams; U.S. Pat. No. 6,953,040 to Atchley et al.; U.S. Pat. No. 7,032,601 to Atchley et al.; and U.S. Pat. No. 7,694,686 to Atchley et al.; US Pat. Pub. Nos. 2004/0020503 to Williams; 2005/0115580 to Quinter et al.; 2006/0191548 to Strickland et al.; 2007/0062549 to Holton, Jr. et al.; 2007/0186941 to Holton, Jr. et al.; 2007/0186942 to Strickland et al.; 2008/0029110 to Dube et al.; 2008/0029116 to Robinson et al.; 2008/0173317 to Robinson et al.; 2008/0209586 to Neilsen et al.; 2009/0065013 to Essen et al.; and 2010/0282267 to Atchley, as well as WO2004/095959 to Arnarp et al., each of which is incorporated herein by reference.

Smokeless tobacco product configurations that combine tobacco material with various binders and fillers have been proposed more recently, with example product formats including lozenges, pastilles, gels, extruded forms, and the like. See, for example, the types of products described in US Patent App. Pub. Nos. 2008/0196730 to Engstrom et al.; 2008/0305216 to Crawford et al.; 2009/0293889 to Kumar et al.; 2010/0291245 to Gao et al; 2011/0139164 to Mua et al.; 2012/0037175 to Cantrell et al.; 2012/0055494 to Hunt et al.; 2012/0138073 to Cantrell et al.; 2012/0138074 to Cantrell et al.; 2013/0074855 to Holton, Jr.; 2013/0074856 to Holton, Jr.; 2013/0152953 to Mua et al.; 2013/0274296 to Jackson et al.; 2015/0068545 to Moldoveanu et al.; 2015/0101627 to Marshall et al.; and 2015/0230515 to Lampe et al., each of which is incorporated herein by reference.

All-white snus portions are growing in popularity, and offer a discrete and aesthetically pleasing alternative to traditional snus. Such modern “white” pouched products may include a bleached tobacco or may be tobacco-free.

It would be desirable to provide products configured for oral use which may deliver active ingredients to the consumer in an enjoyable form, such as in the form of a pouched product.

### BRIEF SUMMARY

In accordance with some embodiments described herein, there is provided a process for preparing a pouched oral product, wherein the pouched oral product comprises a saliva permeable pouch and an oral product incorporated within the pouch, the process comprising:
(a) providing the oral product,
(b) incorporating the oral product within the pouch to provide a pouched oral product; and
(c) applying a cannabinoid to the surface of the pouch or applying a cannabinoid to the oral product through the pouch.

In accordance with some embodiments described herein, there is provided a process for preparing a pouched oral product, wherein the pouched oral product comprises a saliva permeable pouch and an oral product incorporated within the pouch, the process comprising:
(a) providing the oral product, and
(b) incorporating the oral product within the pouch to provide a pouched oral product;
   wherein the pouch material contains a cannabinoid or has a cannabinoid on its outer surface.

In accordance with some embodiments described herein, there is provided a pouched oral product, wherein the pouched oral product comprises a saliva permeable pouch and an oral product incorporated within the pouch, wherein the pouch material contains a cannabinoid or has a cannabinoid on its outer surface.

In accordance with some embodiments described herein, there is provided a pouched oral product obtained or obtainable by a process as defined herein.

In accordance with some embodiments described herein, there is provided a package containing at least one pouched oral product as defined herein.

The disclosure includes, without limitations, the following embodiments.

Embodiment 1: A process for preparing a pouched oral product, wherein the pouched oral product comprises a saliva permeable pouch and an oral product incorporated within the pouch, the process comprising: (a) providing the oral product, (b) incorporating the oral product within the pouch to provide a pouched oral product; and (c) applying a cannabinoid to the surface of the pouch or applying a cannabinoid to the oral product through the pouch.

Embodiment 2: A process according to embodiment 1, wherein (c) comprises applying a cannabinoid to the surface of the pouch.

Embodiment 3: A process according to embodiment 2, wherein (c) comprises applying the cannabinoid to the pouch via spraying, dropping or spreading the cannabinoid onto the pouch, or via immersing the pouch in a substance comprising the cannabinoid.

Embodiment 4: A process according to embodiment 1, wherein (c) comprises applying the cannabinoid to the oral product through the pouch.

Embodiment 5: A process according to embodiment 4, wherein (c) comprises applying the cannabinoid to the oral product through the pouch via injection.

Embodiment 6: A process according to any one of embodiments 1 to 5, wherein the pouch comprises a nonwoven fleece material.

Embodiment 7: A process according to embodiment 6, wherein the pouch comprises viscose.

Embodiment 8: A process according to any one of embodiments 1 to 7, wherein the cannabinoid is selected from the group consisting of cannabigerol (CBG), cannabichromene (CBC), cannabidiol (CBD), tetrahydrocannabinol (THC), cannabinol (CBN) and cannabinodiol (CBDL), cannabicyclol (CBL), cannabivarin (CBV), tetrahydrocannabivarin (THCV), cannabidivarin (CBDV), cannabichromevarin (CBCV), cannabigerovarin (CBGV), cannabigerol monomethyl ether (CBGM), cannabinerolic acid, cannabidiolic acid (CBDA), Cannabinol propyl variant (CBNV), cannabitriol (CBO), tetrahydrocannabmolic acid (THCA), tetrahydrocannabivarinic acid (THCV A), and mixtures thereof.

Embodiment 9: A process according to any one of embodiments 1 to 8, wherein the cannabinoid comprises cannabidiol.

Embodiment 10: A process according to embodiment 9, wherein the cannabinoid comprises cannabidiol in an amount of at least 98% by weight of the cannabinoid.

Embodiment 11: A process according to any one of embodiments 1 to 10, wherein the oral product provided in (a) does not contain any cannabinoid.

Embodiment 12: A process according to any one of embodiments 1 to 11, wherein the oral product provided in (a) comprises a cannabinoid.

Embodiment 13: A process according to embodiment 12, wherein the oral product provided in (a) comprises a cannabinoid contained in an emulsion that comprises a dispersed phase and a continuous phase.

Embodiment 14: A process according to any one of embodiments 1 to 13, wherein the oral product provided in (a) comprises a filler.

Embodiment 15: A process according to embodiment 14, wherein the filler is a cellulose material selected from the group consisting of maize fiber, oat fiber, barley fiber, rye fiber, buckwheat fiber, sugar beet fiber, bran fiber, bamboo fiber, wood pulp fiber, cotton fiber, citrus pulp fiber, grass fiber, willow fiber, poplar fiber, cocoa fiber, derivatives thereof, and combinations thereof.

Embodiment 16: A process for preparing a pouched oral product, wherein the pouched oral product comprises a saliva permeable pouch and an oral product incorporated within the pouch, the process comprising: (a) providing the oral product, and (b) incorporating the oral product within the pouch to provide a pouched oral product; wherein the pouch material contains a cannabinoid or has a cannabinoid on its outer surface.

Embodiment 17: A process according to embodiment 16, wherein the cannabinoid is applied to the pouch via spraying, dropping, or spreading the cannabinoid onto the pouch, or via immersing the pouch in a substance comprising the cannabinoid.

Embodiment 18: A pouched oral product, wherein the pouched oral product comprises a saliva permeable pouch and an oral product incorporated within the pouch, wherein the pouch material contains a cannabinoid or has a cannabinoid on its outer surface.

Embodiment 19: A pouched oral product according to embodiment 18, wherein the oral product incorporated within the pouch does not comprise any cannabinoid.

Embodiment 20: A pouched oral product according to embodiment 18, wherein the oral product incorporated within the pouch further comprises a cannabinoid.

Embodiment 21: A pouched oral product according to any one of embodiments 18 to 20, wherein the total amount of cannabinoid in the pouched oral product is from about 0.5% to about 20% of the pouched oral product.

Embodiment 22: A pouched oral product obtained or obtainable by a process as defined in any one of embodiments 1 to 17.

Embodiment 23: A package containing at least one pouched oral product as defined in any one of embodiments 18 to 22.

Embodiment 24: A process, product, or package according to any one of embodiments 1 to 23, wherein the cannabinoid is replaced in whole or in part with a cannabimimetic.

These and other features, aspects, and advantages of the disclosure will be apparent from a reading of the following detailed description together with the accompanying drawings, which are briefly described below. The invention includes any combination of two, three, four, or more of the above-noted embodiments as well as combinations of any two, three, four, or more features or elements set forth in this disclosure, regardless of whether such features or elements are expressly combined in a specific embodiment description herein. This disclosure is intended to be read holistically such that any separable features or elements of the disclosed invention, in any of its various aspects and embodiments, should be viewed as intended to be combinable unless the context clearly dictates otherwise.

### BRIEF DESCRIPTION OF THE DRAWINGS

Having thus described aspects of the disclosure in the foregoing general terms, reference will now be made to the accompanying drawings, which are not necessarily drawn to scale. The drawings are exemplary only, and should not be construed as limiting the disclosure. Embodiments of the invention will now be described, by way of example only, with reference to accompanying drawings, in which:

FIG. 1 is a cross-sectional view of a pouched product embodiment, taken across the width of the product, showing an outer pouch filled with a composition as described herein; and

FIG. 2 shows a schematic view of a process according to the present invention.

### DETAILED DESCRIPTION

As described herein, there is provided a process for preparing a pouched oral product, wherein the pouched oral product comprises a saliva permeable pouch and an oral product incorporated within the pouch, the process comprising:
(a) providing the oral product,
(b) incorporating the oral product within the pouch to provide a pouched oral product; and
(c) applying a cannabinoid to the surface of the pouch or applying a cannabinoid to the oral product through the pouch.

Also described herein is a process for preparing a pouched oral product, wherein the pouched oral product comprises a saliva permeable pouch and an oral product incorporated within the pouch, the process comprising:
(a) providing the oral product, and
(b) incorporating the oral product within the pouch to provide a pouched oral product;
   wherein the pouch material contains a cannabinoid or has a cannabinoid on its outer surface.

The present disclosure will now be described more fully hereinafter with reference to example embodiments thereof. These example embodiments are described so that this disclosure will be thorough and complete, and will fully convey the scope of the disclosure to those skilled in the art. Indeed, the disclosure may be embodied in many different forms and should not be construed as limited to the embodiments set forth herein; rather, these embodiments are provided so that this disclosure will satisfy applicable legal requirements.

As used in this specification and the claims, the singular forms “a,” “an,” and “the” include plural referents unless the context clearly dictates otherwise. Reference to “dry weight percent” or “dry weight basis” refers to weight on the basis of dry ingredients (i.e., all ingredients except water). Reference to “wet weight” refers to the weight of the composition including water. Unless otherwise indicated, reference to “weight percent” of a composition reflects the total wet weight of the composition (i.e., including water).

The relative amounts of the various components within the product may vary, and typically are selected so as to provide the desired sensory and performance characteristics to the oral product. The example individual constituents of the composition are described herein below.

### Process

According to some embodiments disclosed herein, there is provided a process for preparing a pouched oral product. The pouched oral product is configured for oral use, and thus for insertion into the user's mouth (i.e., oral cavity).

The pouched oral product comprises a saliva-permeable pouch. The saliva-permeable pouch allows for the passage of saliva through the material that forms the pouch (i.e., “the pouch material”). As such, when the pouched oral product is placed into the oral cavity or mouth of the user, saliva may penetrate the oral product incorporated therein. The saliva may dissolve or disperse a number of the components within the oral product therein, and such saliva-soluble or -dispersable ingredients may then pass through the saliva-permeable pouch material and into the mouth of the user.

The oral product incorporated within the pouch may be in the form of a powder, for example. The pouched oral product may thus be similar to a snus-type product, for example.

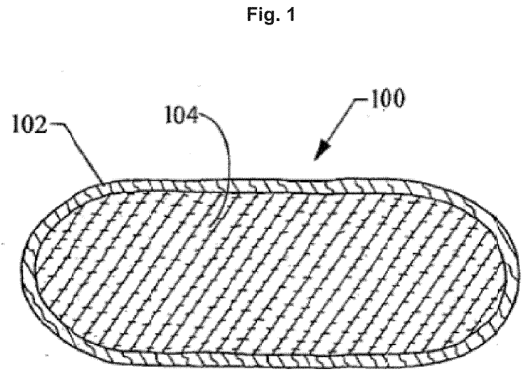

Accordingly, the oral product is combined within a saliva-permeable packet or pouch that acts as a container for use of the composition to provide a pouched product configured for oral use. Certain embodiments of the disclosure will be described with reference to FIG. 1 of the accompanying drawing, and these described embodiments involve snus-type products having an outer pouch and containing a composition as described herein. As explained in greater detail below, such embodiments are provided by way of example only, and the pouched products of the present disclosure can include the composition in other forms. The composition/construction of such packets or pouches, such as the container pouch 102 in the embodiment illustrated in FIG. 1, may be varied. Referring to FIG. 1, there is shown a first embodiment of a pouched product 100. The pouched product 100 includes a moisture-permeable container in the form of a pouch 102, which contains an oral product 104 as described herein.

### Oral Product

In some embodiments, (a) providing the oral product comprises providing an oral product in solid form. A solid oral product is a composition which can substantially sustain its physical shape when unsupported by external means, e.g. packaging etc. Thus, it is considered to be solid, solid like, in solid form or in solid-like form at room temperature. For the avoidance of doubt a solid product must remain substantially solid at up to 30° C. By solid-like, it is understood that some materials are considered on a day to day basis to be solid, yet over an extremely long period of time, may alter in shape, e.g., amorphous materials such as glass etc. However, they are considered to be solid-like as, for the purpose they fulfil, they are solid.

In some embodiments, the oral product as described herein is provided in a solid form. The products may take various forms, including pastilles, gums, lozenges, tablets, and powders. In some embodiments, the oral product is provided in the form of a powder, such as a free-flowing powder.

In some embodiments, the oral product includes an active agent. In some embodiments, the oral product is substantially free of any active agent before incorporation of the oral product into the pouch.

The emulsion and compositions and products comprising the emulsion as described herein are configured for oral use. The term “configured for oral use” as used herein means that the product is provided in a form such that during use, saliva in the mouth of the user causes one or more of the components of the emulsion, composition, or product (e.g., flavoring agents and/or active ingredients) to pass into the mouth of the user. In certain embodiments, the emulsion, composition, or product is adapted to deliver components to a user through mucous membranes in the user's mouth, the user's digestive system, or both, and, in some instances, said component is an active ingredient that can be absorbed through the mucous membranes in the mouth or absorbed through the digestive tract when the product is used.

Oral products configured for oral use as described may be in a solid form. The products may take various forms, including pastilles, gums, lozenges, tablets, and powders. The products may be in the form of a solid oral product (e.g., a powder) which is incorporated within a pouch.

Certain products configured for oral use are in the form of pastilles. As used herein, the term “pastille” refers to a dissolvable oral product made by solidifying a liquid or gel composition so that the final product is a somewhat hardened solid gel. The rigidity of the gel is highly variable. Certain products can exhibit, for example, one or more of the following characteristics: crispy, granular, chewy, syrupy, pasty, fluffy, smooth, and/or creamy. In certain embodiments, the desired textural property can be selected from the group consisting of adhesiveness, cohesiveness, density, dryness, fracturability, graininess, gumminess, hardness, heaviness, moisture absorption, moisture release, mouthcoating, roughness, slipperiness, smoothness, viscosity, wetness, and combinations thereof.

The oral products of the present disclosure may be dissolvable. As used herein, the terms “dissolve,” “dissolving,” and “dissolvable” refer to compositions having aqueous-soluble components that interact with moisture in the oral cavity and enter into solution, thereby causing gradual consumption of the product. According to one aspect, the dissolvable product is capable of lasting in the user's mouth for a given period of time until it completely dissolves. Dissolution rates can vary over a wide range, from about 1 minute or less to about 60 minutes. For example, fast release compositions typically dissolve and/or release the active substance in about 2 minutes or less, often about 1 minute or less (e.g., about 50 seconds or less, about 40 seconds or less, about 30 seconds or less, or about 20 seconds or less). Dissolution can occur by any means, such as melting, mechanical disruption (e.g., chewing), enzymatic or other chemical degradation, or by disruption of the interaction between the components of the composition. In some embodiments, the product can be meltable as discussed, for example, in US Patent App. Pub. No. 2012/0037175 to Cantrell et al. In other embodiments, the products do not dissolve during the product's residence in the user's mouth.

In some embodiments, the oral product may be in the form of a powder. The powder may be a free-flowing powder. The oral product in the form of a powder is then incorporated into a moisture-permeable (e.g., saliva-permeable) pouch, similar to a snus-type product. The pouched product may be configured for insertion into the oral cavity of a user.

### Filler

In some embodiments, the oral product includes a filler. Fillers may fulfil multiple functions, such as enhancing certain organoleptic properties such as texture and mouthfeel, enhancing cohesiveness or compressibility of the product, and the like, depending on the product.

In some embodiments, the filler is a porous particulate material and is cellulose-based. For example, the filler may be a non-tobacco plant material or derivative thereof, including cellulose materials derived from such sources. Examples of cellulosic non-tobacco plant material include cereal grains (e.g., maize, oat, barley, rye, buckwheat, and the like), sugar beet (e.g., FIBREX® brand filler available from International Fiber Corporation), bran fiber, and mixtures thereof.

In some embodiments, the filler is a cellulose material selected from the group consisting of maize fiber, oat fiber, barley fiber, rye fiber, buckwheat fiber, sugar beet fiber, bran fiber, bamboo fiber, wood pulp fiber, cotton fiber, citrus pulp fiber, grass fiber, willow fiber, poplar fiber, cocoa fiber, derivatives thereof, and combinations thereof. In some embodiments, the filler is a cellulose material selected from the group consisting of maize fiber, oat fiber, sugar beet fiber, bamboo fiber, wood pulp fiber, cotton fiber, grass fiber, derivatives thereof, and combinations thereof. In some embodiments, the filler is a cellulose material selected from the group consisting of sugar beet fiber, wood pulp fiber, bamboo fiber, derivatives thereof, and combinations thereof.

In some embodiments, the filler is derived from any of maize fiber, oat fiber, barley fiber, rye fiber, buckwheat fiber, sugar beet fiber, bran fiber, bamboo fiber, wood pulp fiber, cotton fiber, citrus pulp fiber, grass fiber, willow fiber, poplar fiber, cocoa fiber, or combinations thereof. In some embodiments, the filler is derived from wood pulp fiber.

In some embodiments, the filler is a cellulose material. One particularly suitable filler for use in the compositions described herein is microcrystalline cellulose (“MCC”). MCC is typically derived from wood pulp fiber. MCC is composed of glucose units connected by a 1-4 beta glycosidic bond, and may be synthesized by partially depolymerizing alpha-cellulose, by, for example, reactive extrusion, enzyme mediated depolymerisation, mechanical grinding, ultrasonication, steam explosion and/or acid hydrolysis. The MCC may be synthetic or semi-synthetic, or it may be obtained entirely from natural celluloses. The MCC may be selected from the group consisting of AVICEL® grades PH-100, PH-101, PH-102, PH-103, PH-105, PH-112, PH-113, PH-200, PH-300, PH-301, PH-302, VIVACEL® grades 101, 102, 12, 20 and EMOCEL® grades 50M and 90M, and the like, and mixtures thereof. In some embodiments, the oral product comprises MCC as the filler.

In some embodiments, the filler is a non-tobacco plant material or a derivative thereof. Non-limiting examples of derivatives of non-tobacco plant material include starches (e.g., from potato, wheat, rice, corn), natural cellulose, and modified cellulosic materials. Additional examples of potential fillers include maltodextrin, dextrose, calcium carbonate, calcium phosphate, lactose, mannitol, xylitol, and sorbitol. Combinations of fillers can also be used. “Starch” as used herein may refer to pure starch from any source, modified starch, or starch derivatives. Starch is present, typically in granular form, in almost all green plants and in various types of plant tissues and organs (e.g., seeds, leaves, rhizomes, roots, tubers, shoots, fruits, grains, and stems). Starch can vary in composition, as well as in granular shape and size. Often, starch from different sources has different chemical and physical characteristics. A specific starch can be selected for inclusion in the composition based on the ability of the starch material to impart a specific organoleptic property to composition. Starches derived from various sources can be used. For example, major sources of starch include cereal grains (e.g., rice, wheat, and maize) and root vegetables (e.g., potatoes and cassava). Other examples of sources of starch include acorns, arrowroot, arracacha, bananas, barley, beans (e.g., favas, lentils, mung beans, peas, chickpeas), breadfruit, buckwheat, canna, chestnuts, colacasia, katakuri, kudzu, malanga, millet, oats, oca, Polynesian arrowroot, sago, sorghum, sweet potato, quinoa, rye, tapioca, taro, tobacco, water chestnuts, and yams. Certain starches are modified starches. A modified starch has undergone one or more structural modifications, often designed to alter its high heat properties. Some starches have been developed by genetic modifications, and are considered to be “genetically modified” starches. Other starches are obtained and subsequently modified by chemical, enzymatic, or physical means. For example, modified starches can be starches that have been subjected to chemical reactions, such as esterification, etherification, oxidation, depolymerization (thinning) by acid catalysis or oxidation in the presence of base, bleaching, transglycosylation and depolymerization (e.g., dextrinization in the presence of a catalyst), cross-linking, acetylation, hydroxypropylation, and/or partial hydrolysis. Enzymatic treatment includes subjecting native starches to enzyme isolates or concentrates, microbial enzymes, and/or enzymes native to plant materials, e.g., amylase present in corn kernels to modify corn starch. Other starches are modified by heat treatments, such as pregelatinization, dextrinization, and/or cold water swelling processes. Certain modified starches include monostarch phosphate, distarch glycerol, distarch phosphate esterified with sodium trimetaphosphate, phosphate distarch phosphate, acetylated distarch phosphate, starch acetate esterified with acetic anhydride, starch acetate esterified with vinyl acetate, acetylated distarch adipate, acetylated distarch glycerol, hydroxypropyl starch, hydroxypropyl distarch glycerol, and starch sodium octenyl succinate.

The amount of the filler can vary, but when present, is typically at least about 50 percent by weight of the oral product, based on the total weight of the oral product. A typical range of filler (e.g., microcrystalline cellulose) within the composition can be from about 10 to about 75 percent by total weight of the oral product. For example, the filler (e.g., MCC) may be present in the oral product in an amount of at least about 50% by weight of the oral product, such as at least about 55% by weight of the oral product, such as at least about 60% by weight of the oral product. In some embodiments, the filler (e.g., MCC) may be present in the oral product in an amount of from about 50% to about 99% by weight of the oral product, such as from about 50% to about 95% by weight of the oral product, such as from about 50% to about 90% by weight of the oral product, such as from about 55% to about 85% by weight of the oral product, such as from about 60% to about 80% by weight of the oral product, such as from about 60% to about 75% by weight of the oral product.

In some embodiments, the oral product comprises microcrystalline cellulose in an amount of from about 55% to about 95% by weight of the oral product. In some embodiments, the oral product comprises microcrystalline cellulose in an amount of from about 55% to about 80% by weight of the oral product.

For the avoidance of doubt, combinations of the above end points are explicitly envisaged by the present disclosure. This applies to any of the ranges disclosed herein.

### Active Agent

In some embodiments, (a) providing an oral product comprises providing an oral product that includes at least one active agent. In some embodiments, (a) providing an oral product comprises providing an oral product that is substantially free of any active agent. In some embodiments, (a) providing an oral product comprises providing an oral product that does not include any active agent. As referred to herein, “substantially free of any active agent” means that the oral product includes an active agent in an amount of no greater than about 1% by weight, such as no greater than about 0.5% by weight, such as no greater than about 0.1% by weight, such as no greater than about 0.01% by weight of the oral product.

In some embodiments, (a) comprises providing an oral product that includes at least one active agent. In some embodiments, two or more active ingredients can be incorporated within the same oral product.

In some embodiments, the oral product may comprise an active agent in an amount of from about 0.01% to about 50% by weight, such as from about 0.1% to about 40% by weight, such as from about 1% to about 30% by weight, such as from about 5% to about 25% by weight, such as from about 10% to about 20% by weight, such as from about 10% to about 15% by weight of the oral product.

As used herein, an “active agent” or “active ingredient” refers to one or more substances belonging to any of the following categories: API (active pharmaceutical substances), food additives, natural medicaments, and naturally occurring substances that can have an effect on humans. Example active ingredients include any ingredient known to impact one or more biological functions within the body, such as ingredients that furnish pharmacological activity or other direct effect in the diagnosis, cure, mitigation, treatment, or prevention of disease, or which affect the structure or any function of the body of humans (e.g., provide a stimulating action on the central nervous system, have an energizing effect, an antipyretic or analgesic action, or an otherwise useful effect on the body). In some embodiments, the active ingredient may be of the type generally referred to as dietary supplements, nutraceuticals, “phytochemicals” or “functional foods”. These types of additives are sometimes defined in the art as encompassing substances typically available from naturally-occurring sources (e.g., botanical materials) that provide one or more advantageous biological effects (e.g., health promotion, disease prevention, or other medicinal properties), but are not classified or regulated as drugs.

Non-limiting examples of active ingredients include those falling in the categories of botanical ingredients (e.g., hemp, lavender, peppermint, eucalyptus, rooibos, fennel, cloves, chamomile, basil, rosemary, clove, citrus, ginger, cannabis, ginseng, maca, and tisanes), stimulants (e.g., caffeine or guarana), amino acids (e.g., taurine, theanine, phenylalanine, tyrosine, and tryptophan), vitamins (e.g., B12, and C), antioxidants, nicotine components, pharmaceutical ingredients (e.g., nutraceutical and medicinal ingredients), and/or melatonin. Each of these categories is further described herein below. The particular choice of active ingredients will vary depending upon the desired flavor, texture, and desired characteristics of the particular product.

The particular percentages of active ingredients present will vary depending upon the desired characteristics of the particular product. Typically, an active ingredient or combination thereof is present in a total concentration of at least about 0.001% by weight of the composition, such as in a range from about 0.001% to about 20%. In some embodiments, the active ingredient or combination of active ingredients is present in a concentration from about 0.1% w/w to about 10% by weight, such as, e.g., from about 0.5% w/w to about 10%, from about 1% to about 10%, from about 1% to about 5% by weight, based on the total weight of the composition. In some embodiments, the active ingredient or combination of active ingredients is present in a concentration of from about 0.001%, about 0.01%, about 0.1%, or about 1%, up to about 20% by weight, such as, e.g., from about 0.001%, about 0.002%, about 0.003%, about 0.004%, about 0.005%, about 0.006%, about 0.007%, about 0.008%, about 0.009%, about 0.01%, about 0.02%, about 0.03%, about 0.04%, about 0.05%, about 0.06%, about 0.07%, about 0.08%, about 0.09%, about 0.1%, about 0.2%, about 0.3%, about 0.4%, about 0.5% about 0.6%, about 0.7%, about 0.8%, or about 0.9%, to about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, or about 20% by weight, based on the total weight of the composition. Further suitable ranges for specific active ingredients are provided herein below.

Botanical

In some embodiments, (a) providing an oral product comprises providing an oral product that comprises a botanical ingredient as an active agent. As used herein, the term “botanical ingredient” or “botanical” refers to any plant material or fungal-derived material, including plant material in its natural form and plant material derived from natural plant materials, such as extracts or isolates from plant materials or treated plant materials (e.g., plant materials subjected to heat treatment, fermentation, bleaching, or other treatment processes capable of altering the physical and/or chemical nature of the material). For the purposes of the present disclosure, a “botanical” includes, but is not limited to, “herbal materials,” which refer to seed-producing plants that do not develop persistent woody tissue and are often valued for their medicinal or sensory characteristics (e.g., teas or tisanes). Reference to botanical material as “non-tobacco” is intended to exclude tobacco materials (i.e., does not include any Nicotiana species).

When present, a botanical is typically at a concentration of from about 0.01% w/w to about 10% by weight, such as, e.g., from about 0.01% w/w, about 0.05%, about 0.1%, or about 0.5%, to about 1%, about 2%, about 3%, about 4%, about 5%, about 6%, about 7%, about 8%, about 9%, or about 10%, about 11%, about 12%, about 13%, about 14%, or about 15% by weight, based on the total weight of the composition.

The botanical materials useful in the present disclosure may comprise, without limitation, any of the compounds and sources set forth herein, including mixtures thereof. Certain botanical materials of this type are sometimes referred to as dietary supplements, nutraceuticals, “phytochemicals” or “functional foods.” Certain botanicals, as the plant material or an extract thereof, have found use in traditional herbal medicine, and are described further herein. Non-limiting examples of botanicals or botanical-derived materials include hemp, eucalyptus, rooibos, fennel, citrus, cloves, lavender, peppermint, chamomile, basil, rosemary, ginger, turmeric, green tea, white mulberry, cannabis, cocoa, ashwagandha, baobab, chlorophyll, cordyceps, damiana, ginseng, guarana, and maca. In some embodiments, the composition comprises green tea, turmeric, and white mulberry.

Ashwagandha (Withania somnifera) is a plant in the Solanaceae (nightshade) family. As an herb, Ashwagandha has found use in the Indian Ayurvedic system of medicine, where it is also known as “Indian Winter cherry” or “Indian Ginseng.” In some embodiments, the active ingredient comprises ashwagandha.

Baobab is the common name of a family of deciduous trees of the genus Adansonia. The fruit pulp and seeds of the Baobab are consumed, generally after drying, as a food or nutritional supplement. In some embodiments, the active ingredient comprises baobab.

Chlorophyll is any of several related green pigments found in the mesosomes of cyanobacteria, as well as in the chloroplasts of algae and plants. Chlorophyll has been used as a food additive (colorant) and a nutritional supplement. Chlorophyll may be provided either from native plant materials (e.g., botanicals) or in an extract or dried powder form. In some embodiments, the active ingredient comprises chlorophyll.

Cordyceps is a diverse genus of ascomycete (sac) fungi which are abundant in humid temperate and tropical forests. Members of the cordyceps family are used extensively in traditional Chinese medicine. In some embodiments, the active ingredient comprises cordyceps.

Damiana is a small, woody shrub of the family Passifloraceae. It is native to southern Texas, Central America, Mexico, South America, and the Caribbean. Damiana produces small, aromatic flowers, followed by fruits that taste similar to Figs. The extract from damiana has been found to suppress aromatase activity, including the isolated compounds pinocembrin and acacetin. In some embodiments, the active ingredient comprises damiana.

Guarana is a climbing plant in the family Sapindaceae, native to the Amazon basin. The seeds from its fruit, which are about the size of a coffee bean, have a high concentration of caffeine and, consequently, stimulant activity. In some embodiments, the active ingredient comprises guarana. In some embodiments, the active ingredient comprises guarana, honey, and ashwagandha.

Ginseng is the root of plants of the genus Panax, which are characterized by the presence of unique steroid saponin phytochemicals (ginsenosides) and gintonin. Ginseng finds use as a dietary supplement in energy drinks or herbal teas, and in traditional medicine. Cultivated species include Korean ginseng (P. ginseng), South China ginseng (P. notoginseng), and American ginseng (P. quinquefolius). American ginseng and Korean ginseng vary in the type and quantity of various ginsenosides present. In some embodiments, the active ingredient comprises ginseng. In some embodiments, the ginseng is American ginseng or Korean ginseng. In specific embodiments, the active ingredient comprises Korean ginseng.

Maca is a plant that grows in central Peru in the high plateaus of the Andes Mountains. It is a relative of the radish, and has an odor similar to butterscotch. Maca has been used in traditional (e.g., Chinese) medicine. In some embodiments, the active ingredient comprises maca.

In some embodiments, the botanical may be selected from the group consisting of lavender, peppermint, chamomile, basil, rosemary, ginger, turmeric, green tea, white mulberry, cannabis, cocoa, ashwagandha, baobab, chlorophyll, cordyceps, damiana, ginseng, guarana, maca, and mixtures thereof. In some embodiments, the botanical comprises green tea, turmeric, white mulberry, or mixtures thereof.

### Stimulants

In some embodiments, (a) providing an oral product comprises providing an oral product that comprises a stimulant as an active agent. As used herein, the term “stimulant” refers to a material that increases activity of the central nervous system and/or the body, for example, enhancing focus, cognition, vigor, mood, alertness, and the like. Non-limiting examples of stimulants include caffeine, theacrine, theobromine, and theophylline. Theacrine (1,3,7,9-tetramethyluric acid) is a purine alkaloid which is structurally related to caffeine, and possesses stimulant, analgesic, and anti-inflammatory effects. Present stimulants may be natural, naturally derived, or wholly synthetic. For example, certain botanical materials (guarana, tea, coffee, cocoa, and the like) may possess a stimulant effect by virtue of the presence of e.g., caffeine or related alkaloids, and accordingly are “natural” stimulants. By “naturally derived” is meant the stimulant (e.g., caffeine, theacrine) is in a purified form, outside its natural (e.g., botanical) matrix. For example, caffeine can be obtained by extraction and purification from botanical sources (e.g., tea). By “wholly synthetic”, it is meant that the stimulant has been obtained by chemical synthesis.

When present, a stimulant or combination of stimulants (e.g., caffeine, theacrine, and combinations thereof) is typically at a concentration of from about 0.1% w/w to about 15% by weight, such as, e.g., from about 0.1% w/w, about 0.2%, about 0.3%, about 0.4%, about 0.5% about 0.6%, about 0.7%, about 0.8%, or about 0.9%, to about 1%, about 2%, about 3%, about 4%, about 5%, about 6%, about 7%, about 8%, about 9%, about 10%, about 11%, about 12%, about 13%, about 14%, or about 15% by weight, based on the total weight of the composition.

In some embodiments, the active ingredient comprises caffeine. In some embodiments, the active ingredient comprises theacrine. In some embodiments, the active ingredient comprises a combination of caffeine and theacrine.

### Amino Acids

In some embodiments, (a) providing an oral product comprises providing an oral product that comprises an amino acid as an active agent. As used herein, the term “amino acid” refers to an organic compound that contains amine (—NH₂) and carboxyl (—COOH) or sulfonic acid (SO₃H) functional groups, along with a side chain (R group), which is specific to each amino acid. Amino acids may be proteinogenic or non-proteinogenic. By “proteinogenic” is meant that the amino acid is one of the twenty naturally occurring amino acids found in proteins. The proteinogenic amino acids include alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine. By “non-proteinogenic” is meant that either the amino acid is not found naturally in protein, or is not directly produced by cellular machinery (e.g., is the product of post-tranlational modification). Non-limiting examples of non-proteinogenic amino acids include gamma-aminobutyric acid (GABA), taurine (2-aminoethanesulfonic acid), theanine (L-γ-glutamylethylamide), hydroxyproline, and beta-alanine.

When present, an amino acid or combination of amino acids (e.g., taurine, theanine, and combinations thereof) is typically at a concentration of from about 0.1% w/w to about 15% by weight, such as, e.g., from about 0.1% w/w, about 0.2%, about 0.3%, about 0.4%, about 0.5% about 0.6%, about 0.7%, about 0.8%, or about 0.9%, to about 1%, about 2%, about 3%, about 4%, about 5%, about 6%, about 7%, about 8%, about 9%, about 10%, about 11%, about 12%, about 13%, about 14%, or about 15% by weight, based on the total weight of the composition.

In some embodiments, the amino acid is taurine, theanine, phenylalanine, tyrosine, tryptophan, or a combination thereof. In some embodiments, the amino acid is taurine. In some embodiments, the active ingredient comprises a combination of taurine and caffeine. In some embodiments, the active ingredient comprises a combination of taurine, caffeine, and guarana. In some embodiments, the active ingredient comprises a combination of taurine, maca, and cordyceps. In some embodiments, the active ingredient comprises a combination of theanine and caffeine.

### Vitamins

In some embodiments, (a) providing an oral product comprises providing an oral product that comprises a vitamin as an active agent. As used herein, the term “vitamin” refers to an organic molecule (or related set of molecules) that is an essential micronutrient needed for the proper functioning of metabolism in a mammal. There are thirteen vitamins required by human metabolism, which are: vitamin A (as all-trans-retinol, all-trans-retinyl-esters, as well as all-trans-beta-carotene and other provitamin A carotenoids), vitamin B1 (thiamine), vitamin B2 (riboflavin), vitamin B3 (niacin), vitamin B5 (pantothenic acid), vitamin B6 (pyridoxine), vitamin B7 (biotin), vitamin B9 (folic acid or folate), vitamin B12 (cobalamins), vitamin C (ascorbic acid), vitamin D (calciferols), vitamin E (tocopherols and tocotrienols), and vitamin K (quinones).

When present, a vitamin or combination of vitamins (e.g., vitamin B6, vitamin B12, vitamin E, vitamin C, or a combination thereof) is typically at a concentration of from about 0.01% w/w to about 1% by weight, such as, e.g., from about 0.01%, about 0.02%, about 0.03%, about 0.04%, about 0.05%, about 0.06%, about 0.07%, about 0.08%, about 0.09%, or about 0.1% w/w, to about 0.2%, about 0.3%, about 0.4%, about 0.5% about 0.6%, about 0.7%, about 0.8%, about 0.9%, or about 1% by weight, based on the total weight of the composition.

In some embodiments, the vitamin is vitamin B6, vitamin B12, vitamin E, vitamin C, or a combination thereof. In some embodiments, the active ingredient comprises a combination of vitamin B6, caffeine, and theanine. In some embodiments, the active ingredient comprises vitamin B6, vitamin B12, and taurine. In some embodiments, the active ingredient comprises a combination of vitamin B6, vitamin B12, ginseng, and theanine. In some embodiments, the active ingredient comprises a combination of vitamin C, baobab, and chlorophyll.

In certain embodiments, the active ingredient is selected from the group consisting of caffeine, taurine, GABA, theanine, vitamin C, lemon balm extract, ginseng, citicoline, sunflower lecithin, and combinations thereof. For example, the active ingredient can include a combination of caffeine, theanine, and optionally ginseng. In another embodiment, the active ingredient includes a combination of theanine, gamma-amino butyric acid (GABA), and lemon balm extract. In a further embodiment, the active ingredient includes theanine, theanine and tryptophan, or theanine and one or more B vitamins (e.g., vitamin B6 or B12). In a still further embodiment, the active ingredient includes a combination of caffeine, taurine, and vitamin C

### Antioxidants

In some embodiments, (a) providing an oral product comprises providing an oral product that comprises an antioxidant as an active agent. As used herein, the term “antioxidant” refers to a substance which prevents or suppresses oxidation by terminating free radical reactions, and may delay or prevent some types of cellular damage. Antioxidants may be naturally occurring or synthetic. Naturally occurring antioxidants include those found in foods and botanical materials. Non-limiting examples of antioxidants include certain botanical materials, vitamins, polyphenols, and phenol derivatives.

Examples of botanical materials which are associated with antioxidant characteristics include without limitation acai berry, alfalfa, allspice, annatto seed, apricot oil, basil, bee balm, wild bergamot, black pepper, blueberries, borage seed oil, bugleweed, cacao, calamus root, catnip, catuaba, cayenne pepper, chaga mushroom, chervil, cinnamon, dark chocolate, potato peel, grape seed, ginseng, gingko biloba, Saint John's Wort, saw palmetto, green tea, black tea, black cohosh, cayenne, chamomile, cloves, cocoa powder, cranberry, dandelion, grapefruit, honeybush, echinacea, garlic, evening primrose, feverfew, ginger, goldenseal, hawthorn, hibiscus flower, jiaogulan, kava, lavender, licorice, marjoram, milk thistle, mints (menthe), oolong tea, beet root, orange, oregano, papaya, pennyroyal, peppermint, red clover, rooibos (red or green), rosehip, rosemary, sage, clary sage, savory, spearmint, spirulina, slippery elm bark, sorghum bran hi-tannin, sorghum grain hi-tannin, sumac bran, comfrey leaf and root, goji berries, gutu kola, thyme, turmeric, uva ursi, valerian, wild yam root, wintergreen, yacon root, yellow dock, yerba mate, yerba santa, bacopa monniera, withania somnifera, Lion's mane, and silybum marianum. Such botanical materials may be provided in fresh or dry form, essential oils, or may be in the form of an extracts. The botanical materials (as well as their extracts) often include compounds from various classes known to provide antioxidant effects, such as minerals, vitamins, isoflavones, phytoesterols, allyl sulfides, dithiolthiones, isothiocyanates, indoles, lignans, flavonoids, polyphenols, and carotenoids. Examples of compounds found in botanical extracts or oils include ascorbic acid, peanut endocarb, resveratrol, sulforaphane, beta-carotene, lycopene, lutein, co-enzyme Q, carnitine, quercetin, kaempferol, and the like. See, e.g., Santhosh et al., Phytomedicine, 12(2005) 216-220, which is incorporated herein by reference.

Non-limiting examples of other suitable antioxidants include citric acid, Vitamin E or a derivative thereof, a tocopherol, epicatechol, epigallocatechol, epigallocatechol gallate, erythorbic acid, sodium erythorbate, 4-hexylresorcinol, theaflavin, theaflavin monogallate A or B, theaflavin digallate, phenolic acids, glycosides, quercitrin, isoquercitrin, hyperoside, polyphenols, catechols, resveratrol s, oleuropein, butylated hydroxyanisole (BHA), butylated hydroxytoluene (BHT), tertiary butylhydroquinone (TBHQ), and combinations thereof. In some embodiments, the antioxidant is Vitamin E or a derivative thereof, a flavonoid, a polyphenol, a carotenoid, or a combination thereof.

When present, an antioxidant is typically at a concentration of from about 0.001% w/w to about 10% by weight, such as, e.g., from about 0.001%, about 0.005%, about 0.01% w/w, about 0.05%, about 0.1%, or about 0.5%, to about 1%, about 2%, about 3%, about 4%, about 5%, about 6%, about 7%, about 8%, about 9%, or about 10%, based on the total weight of the composition.

### Terpenes

Active ingredients suitable for use in the present disclosure can also be classified as terpenes, many of which are associated with biological effects, such as calming effects. Terpenes are understood to have the general formula of (C₅H₈)ₙ and include monoterpenes, sesquiterpenes, and diterpenes. Terpenes can be acyclic, monocyclic or bicyclic in structure. Some terpenes provide an entourage effect when used in combination with cannabinoids or cannabimimetics. Examples include beta-caryophyllene, linalool, limonene, beta-citronellol, linalyl acetate, pinene (alpha or beta), geraniol, carvone, eucalyptol, menthone, iso-menthone, piperitone, myrcene, beta-bourbonene, and germacrene, which may be used singly or in combination.

### Pharmaceutical Ingredients

The pharmaceutical ingredient can be any known agent adapted for therapeutic, prophylactic, or diagnostic use. These can include, for example, synthetic organic compounds, proteins and peptides, polysaccharides and other sugars, lipids, inorganic compounds, and nucleic acid sequences, having therapeutic, prophylactic, or diagnostic activity. Non-limiting examples of pharmaceutical ingredients include analgesics and antipyretics (e.g., acetylsalicylic acid, acetaminophen, 3-(4-isobutylphenyl)propanoic acid).

### Nicotine Component

In certain embodiments, (a) providing an oral product comprises providing an oral product that comprises nicotine or a nicotine component. By “nicotine component” is meant any suitable form of nicotine (e.g., free base or salt) for providing oral absorption of at least a portion of the nicotine present. Typically, the nicotine component is selected from the group consisting of nicotine free base and a nicotine salt. In some embodiments, nicotine is in its free base form, which can be easily adsorbed in for example, a microcrystalline cellulose material to form a microcrystalline cellulose-nicotine carrier complex. See, for example, the discussion of nicotine in free base form in US Pat. Pub. No. 2004/0191322 to Hansson, which is incorporated herein by reference.

In some embodiments, at least a portion of the nicotine can be employed in the form of a salt. Salts of nicotine can be provided using the types of ingredients and techniques set forth in U.S. Pat. No. 2,033,909 to Cox et al. and Perfetti, Beitrage Tabakforschung Int., 12: 43-54 (1983), which are incorporated herein by reference. Further salts are disclosed in, for example, U.S. Pat. No. 9,738,622 to Dull et al., and US Pat. Pub. Nos. 2018/0230126 to Dull et al., 2016/0185750 to Dull et al., and 2018/0051002 to Dull et al., each of which is incorporated herein by reference. Additionally, salts of nicotine are available from sources such as Pfaltz and Bauer, Inc. and K&K Laboratories, Division of ICN Biochemicals, Inc. Typically, the nicotine component is selected from the group consisting of nicotine free base, a nicotine salt such as hydrochloride, dihydrochloride, monotartrate, bitartrate, sulfate, salicylate, and nicotine zinc chloride.

In some embodiments, at least a portion of the nicotine can be in the form of a resin complex of nicotine, where nicotine is bound in an ion-exchange resin, such as nicotine polacrilex, which is nicotine bound to, for example, a polymethacrilic acid, such as Amberlite IRP64, Purolite C115HMR, or Doshion P551. See, for example, U.S. Pat. No. 3,901,248 to Lichtneckert et al., which is incorporated herein by reference. Another example is a nicotine-polyacrylic carbomer complex, such as with Carbopol 974P. In some embodiments, nicotine may be present in the form of a nicotine polyacrylic complex.

Typically, the nicotine component (calculated as the free base) when present, is in a concentration of at least about 0.001% by weight of the oral product, such as in a range from about 0.001% to about 10%. In some embodiments, the nicotine component is present in a concentration from about 0.1% to about 10% by weight, such as from about from about 0.1%, about 0.2%, about 0.3%, about 0.4%, about 0.5% about 0.6%, about 0.7%, about 0.8%, or about 0.9%, to about 1%, about 2%, about 3%, about 4%, about 5%, about 6%, about 7%, about 8%, about 9%, or about 10%, calculated as the free base and based on the total weight of the oral product. In some embodiments, the nicotine component is present in a concentration from about 0.1% to about 3% by weight, such as from about from about 0.1% to about 2.5%, such as from about 0.1% to about 2.0%, such as from about 0.1% to about 1.5%, such as from about 0.1% to about 1% by weight, calculated as the free base and based on the total weight of the oral product. These ranges can also apply to other additional active ingredients noted herein.

In some embodiments, the oral product of the disclosure can be characterized as completely free or substantially free of nicotine. For example, certain embodiments can be characterized as having less than 0.1% by weight, or less than 0.01% by weight, or less than 0.001% by weight of a nicotine component, or 0% by weight of a nicotine component, based on the total weight of the oral product.

In some embodiments, (a) providing an oral product comprises providing an oral product that is substantially free of cannabinoids. In some embodiments, (a) providing an oral product comprises providing an oral product that does not include any cannabinoid(s). As referred to herein, “substantially free of any cannabinoids” means that the oral product includes a cannabinoid in an amount of no greater than about 1% by weight, such as no greater than about 0.5% by weight, such as no greater than about 0.1% by weight, such as no greater than about 0.01% by weight of the oral product.

In some embodiments, however, (a) providing an oral product may comprises providing an oral product that does already contain at least one cannabinoid prior to step (b) incorporating the oral product into the pouch. In some embodiments, the oral product comprises a cannabinoid in a concentration of at least about 0.001% by weight of the oral product, such as in a range from about 0.001% to about 20% by weight of the oral product. In some embodiments, the cannabinoid is present in the oral product in a concentration of from about 0.1% to about 15% by weight, based on the total weight of the oral product. In some embodiments, the cannabinoid is present in a concentration from about 1% to about 15% by weight, such as from about from about 5% to about 15% by weight, based on the total weight of the oral product. In some embodiments, the cannabinoid is present in the oral product in a concentration of from about 0.5% to about 10% by weight, such as from about 1% to about 7.5% by weight, such as from 1.5% to about 5% by weight, such as from about 1.5% to about 2.5% by weight, based on the total weight of the oral product.

Cannabinoids are a class of natural or synthetic chemical compounds which act on cannabinoid receptors (i.e., CB1 and CB2) in cells that repress neurotransmitter release in the brain. Cannabinoids are cyclic molecules exhibiting particular properties such as the ability to easily cross the blood-brain barrier. Cannabinoids may be naturally occurring (Phytocannabinoids) from plants such as cannabis, (endocannabinoids) from animals, or artificially manufactured (synthetic cannabinoids). Cannabis species express at least 85 different phytocannabinoids, and these may be divided into subclasses, including cannabigerols, cannabichromenes, cannabidiols, tetrahydrocannabinols, cannabinols and cannabinodiols, and other cannabinoids, such as cannabigerol (CBG), cannabichromene (CBC), cannabidiol (CBD), tetrahydrocannabinol (THC), cannabinol (CBN) and cannabinodiol (CBDL), cannabicyclol (CBL), cannabivarin (CBV), tetrahydrocannabivarin (THCV), cannabidivarin (CBDV), cannabichromevarin (CBCV), cannabigerovarin (CBGV), cannabigerol monomethyl ether (CBGM), cannabinerolic acid, cannabidiolic acid (CBDA), Cannabinol propyl variant (CBNV), cannabitriol (CBO), tetrahydrocannabmolic acid (THCA), and tetrahydrocannabivarinic acid (THCV A).

In some embodiments, the cannabinoid is selected from the group consisting of cannabigerol (CBG), cannabichromene (CBC), cannabidiol (CBD), tetrahydrocannabinol (THC), cannabinol (CBN) and cannabinodiol (CBDL), cannabicyclol (CBL), cannabivarin (CBV), tetrahydrocannabivarin (THCV), cannabidivarin (CBDV), cannabichromevarin (CBCV), cannabigerovarin (CBGV), cannabigerol monomethyl ether (CBGM), cannabinerolic acid, cannabidiolic acid (CBDA), Cannabinol propyl variant (CBNV), cannabitriol (CBO), tetrahydrocannabmolic acid (THCA), tetrahydrocannabivarinic acid (THCV A), and mixtures thereof. In some embodiments, the cannabinoid comprises at least tetrahydrocannabinol (THC). In some embodiments, the cannabinoid is tetrahydrocannabinol (THC). In some embodiments, the cannabinoid comprises at least cannabidiol (CBD). In some embodiments, the cannabinoid is cannabidiol (CBD).

In some embodiments, the cannabinoid present in the oral product is cannabidiol (CBD) or a pharmaceutically acceptable salt thereof. In some embodiments, the cannabidiol is synthetic cannabidiol. In some embodiments, the cannabinoid is added to the oral product in the form of an isolate. In some embodiments, the cannabidiol is added to the oral product in the form of an isolate. An isolate is an extract from a plant, such as cannabis, where the active material of interest (in this case the cannabinoid, such as CBD) is present in a high degree of purity, for example greater than 95%, greater than 96%, greater than 97%, greater than 98%, or around 99% purity.

The choice of cannabinoid and the particular percentages thereof which may be present within the disclosed oral product will vary depending upon the desired flavor, texture, and other characteristics of the oral product.

Where present, a cannabinoid (such as cannabidiol) may be present in the oral product in a concentration of at least about 0.001% by weight of the oral product, such as in a range from about 0.001% to about 20% by weight of the oral product. In some embodiments, the cannabinoid (such as cannabidiol) is present in the oral product in a concentration of from about 0.1% to about 15% by weight, based on the total weight of the oral product. In some embodiments, the cannabinoid (such as cannabidiol) is present in a concentration from about 1% to about 15% by weight, such as from about from about 5% to about 15% by weight, based on the total weight of the oral product. In some embodiments, the cannabinoid (such as cannabidiol) is present in the oral product in a concentration of from about 0.5% to about 10% by weight, such as from about 1% to about 7.5% by weight, such as from 1.5% to about 5% by weight, such as from about 1.5% to about 2.5% by weight, based on the total weight of the oral product.

Alternatively, or in addition to the cannabinoid, the oral product can include a cannabimimetic, which is a class of compounds derived from plants other than cannabis that have biological effects on the endocannabinoid system similar to cannabinoids. Examples include yangonin, alpha-amyrin or beta-amyrin (also classified as terpenes), cyanidin, curcumin (tumeric), catechin, quercetin, salvinorin A, N-acylethanolamines, and N-alkylamide lipids. Such compounds can be used in the same amounts and ratios noted herein for cannabinoids.

In some embodiments, (a) providing an oral product may comprise providing an oral product that comprises an active agent (such as cannabinoid) in combination with a filler. The cannabinoid as disclosed herein may be associated with the filler (such as cellulose material) in various ways. For example, the cannabinoid may be disposed on the surface of a filler (such as a cellulose material, such as microcrystalline cellulose), may be dispersed in or impregnated into (e.g., adsorbed or absorbed) the filler, or the filler and the cannabinoid may be present in an oral product without being physically combined or in physical contact (e.g., they may be provided separately and independently within the same product). In some embodiments, the cannabinoid is dispersed in or impregnated into (e.g., adsorbed or absorbed) microcrystalline cellulose. For example, the cannabinoid may be retained within the pores of the microcrystalline cellulose. In some embodiments, the cannabinoid may be disposed on the surface of microcrystalline cellulose.

Where an active agent, such as a cannabinoid is present in the oral product, the weight ratio of the filler (such as microcrystalline cellulose) to active agent (such as cannabinoid, such as cannabidiol) may be from about 5:1 to about 100:1, such as from about 10:1 to about 60:1, such as from about 15:1 to about 50:1, such as from about 20:1 to about 40:1, such as from about 25:1 to about 35:1. In some embodiments, the weight ratio of microcrystalline cellulose to cannabidiol may be from about 5:1 to about 100:1, such as from about 10:1 to about 60:1, such as from about 15:1 to about 50:1, such as from about 20:1 to about 40:1, such as from about 25:1 to about 35:1.

### Water

In some embodiments, (a) providing an oral product comprises providing an oral product that comprises water. Water may be present as, for example, purified or ultrapure water, saline, buffered saline, or a buffered aqueous phase.

In some embodiments, the water content of the oral product is at least about 10% by weight of the oral product. As referred to herein, “the water content” means the total amount of water in the oral product, as included in any form. In some embodiments, the oral product has a water content of from about 10% to about 30% by weight of the oral product, such as from about 10% to about 25% by weight of the oral product, such as from about 10% to about 20% by weight of the oral product, such as from about 11% to about 15% by weight of the oral product. In some embodiments, the oral product has a water content of from about 12% to about 30% by weight of the oral product, such as from about 13% to about 25% by weight of the oral product, such as from about 14% to about 25% by weight of the oral product, such as from about 15% to about 20% by weight of the oral product.

In some embodiments, the oral product is configured such that the water activity is no greater than about 0.85, such as no greater than about 0.8, such as no greater than about 0.75, such as no greater than about 0.7, such as no greater than about 0.6, such as no greater than about 0.5. It was found by the present inventors that, when the water activity of the oral product was reduced to below 0.85, the oral product could be stored for a period of several weeks or months without exhibiting significant microbiological growth.

As described herein, the “water activity” (a_{w}) of the oral product is the partial vapor pressure of the water in the product divided by the standard state partial vapor pressure of water. Water activity may be calculated using the following formula:

$${a}_{w} = \frac{ρ}{{ρ}^{*}}$$

where ρ is the partial vapor pressure of water in the product, and ρ* is the partial vapor pressure of pure water at the same temperature. The water activity may be measured using any known and suitable measurement method in the art. In some embodiments, the water activity is measured using a resistive electrolytic hygrometer. In some embodiments, the water activity is measured using a capacitance hygrometer. In some embodiments, the water activity is measured using a dew point hygrometer. In some embodiments, the water activity is measured using a water activity meter having a tuneable diode laser.

### Further Components

In some embodiments, the oral product provided in (a) may further comprise at least one additive selected from the group consisting of a flavoring agent (or “flavorant”), a taste modifier, a preservative, a humectant, a sweetener, a binder, a buffering agent, salt, and mixtures thereof. The additive(s) may be present in any form within the oral product.

### Flavoring Agent and Taste Modifier

In some embodiments, the oral product further comprises a flavorant. As used herein, the terms “flavor” and “flavorant” refer to materials which, where local regulations permit, may be used to create a desired taste, aroma or other somatosensorial sensation in a product for adult consumers. Examples of sensory characteristics that can be modified by the flavoring agent include taste, mouthfeel, moistness, coolness/heat, and/or fragrance/aroma. Flavoring agents may be natural or synthetic, and the character of the flavors imparted thereby may be described, without limitation, as fresh, sweet, herbal, confectionary, floral, fruity, or spicy. They may include naturally occurring flavor materials, botanicals, extracts of botanicals, synthetically obtained materials, or combinations thereof (e.g., tobacco, cannabis, licorice (liquorice), hydrangea, eugenol, Japanese white bark magnolia leaf, chamomile, fenugreek, clove, maple, matcha, menthol, Japanese mint, aniseed (anise), cinnamon, turmeric, Indian spices, Asian spices, herb, wintergreen, cherry, berry, red berry, cranberry, peach, apple, orange, mango, clementine, lemon, lime, tropical fruit, papaya, rhubarb, grape, durian, dragon fruit, cucumber, blueberry, mulberry, citrus fruits, Drambuie, bourbon, scotch, whiskey, gin, tequila, rum, spearmint, peppermint, lavender, aloe vera, cardamom, celery, cascarilla, nutmeg, sandalwood, bergamot, geranium, khat, naswar, betel, shisha, pine, honey essence, rose oil, vanilla, lemon oil, orange oil, orange blossom, cherry blossom, cassia, caraway, cognac, jasmine, ylang-ylang, sage, fennel, wasabi, piment, ginger, coriander, coffee, hemp, a mint oil from any species of the genus Mentha, eucalyptus, star anise, cocoa, lemongrass, rooibos, flax, Ginkgo biloba, hazel, hibiscus, laurel, mate, orange skin, rose, tea such as green tea or black tea, thyme, juniper, elderflower, basil, bay leaves, cumin, oregano, paprika, rosemary, saffron, lemon peel, mint, beefsteak plant, curcuma, cilantro, myrtle, cassis, valerian, pimento, mace, damien, marjoram, olive, lemon balm, lemon basil, chive, carvi, verbena, tarragon, limonene, thymol, camphene), flavor enhancers, bitterness receptor site blockers, sensorial receptor site activators or stimulators, sugars and/or sugar substitutes (e.g., sucralose, acesulfame potassium, aspartame, saccharine, cyclamates, lactose, sucrose, glucose, fructose, sorbitol, or mannitol), and other additives such as charcoal, chlorophyll, minerals, botanicals, or breath freshening agents. They may be imitation, synthetic or natural ingredients or blends thereof. They may be in any suitable form, for example, liquid such as an oil, solid such as a powder, or gas.

In some embodiments, the flavor comprises menthol, spearmint and/or peppermint. In some embodiments, the flavor comprises flavor components of cucumber, blueberry, citrus fruits and/or redberry. In some embodiments, the flavor comprises eugenol. In some embodiments, the flavor comprises flavor components extracted from tobacco. In some embodiments, the flavor comprises flavor components extracted from cannabis.

In some embodiments, the flavor may comprise a sensate, which is intended to achieve a somatosensorial sensation which are usually chemically induced and perceived by the stimulation of the fifth cranial nerve (trigeminal nerve), in addition to or in place of aroma or taste nerves, and these may include agents providing heating, cooling, tingling, numbing effect. A suitable heat effect agent may be, but is not limited to, vanillyl ethyl ether and a suitable cooling agent may be, but not limited to eucolyptol, WS-3.

In some embodiments, the flavorant is lipophilic. Without wishing to be bound by theory, formulation of a lipophilic flavorant as an emulsion may enhance the stability of the flavorant (e.g., toward oxidation or evaporation). In some embodiments, the flavorant is susceptible to oxidation, meaning exposure to air results in the degradation of components in the flavorant due to chemical changes. Examples of functional groups which may be present in flavorant components exhibiting susceptibility to oxidation include, but are not limited to, alkenes, aldehydes, and/or ketones. In some embodiments, the flavorant comprises a citrus oil. Citrus oils contain, for example, terpene components which may be susceptible to oxidation, evaporation, or both and, thus, may particularly benefit from inclusion within a product in the form of an emulsion as described hereinbelow.

In some embodiments, the flavoring agent may comprise a terpene. In some embodiments, the terpene is a terpene derivable from a phytocannabinoid producing plant, such as a plant from the stain of the Cannabis sativa species, such as hemp. Suitable terpenes in this regard include so-called “C10” terpenes, which are those terpenes comprising 10 carbon atoms, and so-called “C15” terpenes, which are those terpenes comprising 15 carbon atoms. In some embodiments, the oral product comprises more than one terpene. For example, the oral product may comprise one, two, three, four, five, six, seven, eight, nine, ten or more terpenes as defined herein. In some embodiments, the terpene is selected from pinene (alpha and beta), geraniol, linalool, limonene, carvone, eucalyptol, menthone, iso-menthone, piperitone, myrcene, beta-bourbonene, germacrene and mixtures thereof.

The amount of flavorant utilized in the oral product can vary, but is typically up to about 10% by weight, and certain embodiments are characterized by a flavoring agent content of at least about 0.1% by weight, such as about 0.5% to about 10% by weight, about 1 to about 6% by weight, or about 2% to about 5% by weight, based on the total weight of the oral product.

In some embodiments, the oral product comprises a taste modifying agent (or “taste modifier”). In some embodiments, the taste modifier may mask the bitterness of the cannabinoid in the product.

The taste modifying agent may improve the organoleptic properties of an oral product as disclosed herein, and may serve to mask, alter, block, or improve e.g., the flavor of a composition as described herein. Non-limiting examples of such taste modifiers include analgesic or anesthetic herbs, spices, and flavors which produce a perceived cooling (e.g., menthol, eucalyptus, mint), warming (e.g., cinnamon), or painful (e.g., capsaicin) sensation. Certain taste modifiers fall into more than one overlapping category.

In some embodiments, the taste modifier modifies one or more of bitter, sweet, salty, or sour tastes. In some embodiments, the taste modifier targets pain receptors. In some embodiments, the cannabinoid has a bitter taste, and the oral product comprises a taste modifier which masks or blocks the perception of the bitter taste. In some embodiments, the taste modifier is a substance which targets pain receptors (e.g., vanilloid receptors) in the user's mouth to mask e.g., a bitter taste of another component (e.g., the cannabinoid). In some embodiments, the taste modifier is capsaicin.

In some embodiments, the taste modifier is the amino acid gamma-amino butyric acid (GABA), referenced herein above with respect to amino acids. Studies in mice suggest that GABA may serve function(s) in taste buds in addition to synaptic inhibition. See, e.g., Dvoryanchikov et al., J Neurosci. 2011 Apr. 13; 31(15):5782-91. Without wishing to be bound by theory, GABA may suppress the perception of certain tastes, such as bitterness. In some embodiments, the composition comprises caffeine and GABA.

In some embodiments, the taste modifier is adenosine monophosphate (AMP). AMP is a naturally occurring nucleotide substance which can block bitter food flavors or enhance sweetness. It does not directly alter the bitter flavor, but may alter human perception of “bitter” by blocking the associated receptor.

In some embodiments, the taste modifier is lactisole. Lactisole is an antagonist of sweet taste receptors. Temporarily blocking sweetness receptors may accentuate e.g., savory notes.

When present, a representative amount of taste modifier is about 0.01% by weight or more, about 0.1% by weight or more, or about 1.0% by weight or more, but will typically make up less than about 10% by weight of the total weight of the oral product, (e.g., from about 0.01%, about 0.05%, about 0.1%, or about 0.5%, to about 1%, about 5%, or about 10% by weight of the total weight of the oral product).

In some embodiments, the taste modifier selected from the group consisting of an analgesic or anesthetic herb, spice, or flavor which produces a perceived cooling or warming effect, gamma-aminobutyric acid, capsaicin, and adenosine monophosphate. In some embodiments, the taste sensation modified by the taste modifier is bitterness, sweetness, saltiness, or sourness. In some embodiments, the taste sensation is bitterness. In some embodiments, the taste modifier is capsaicin.

### Humectant

In certain embodiments, one or more humectants may be employed in the oral product of the present disclosure. The humectant may be present in an emulsion contained within the oral product, or may be present in the composition separate from an emulsion.

In some embodiments, the oral product comprises a humectant. Examples of humectants include, but are not limited to, glycerine, 1,2-propanediol (propylene glycol), 1,3-propanediol, dipropylene glycol, sorbitol, xylitol, mannitol, and the like. In some embodiments, the oral product comprises a humectant selected from the group consisting of glycerine, propylene glycol, 1,3-propanediol, dipropylene glycol, sorbitol, xylitol, mannitol, and mixtures thereof. In some embodiments, the oral product comprises a humectant selected from the group consisting of glycerine, propylene glycol, and mixtures thereof.

In some embodiments, the humectant is or comprises glycerine. In some embodiments, the oral product comprises glycerine. In some embodiments, the humectant is or comprises propylene glycol. In some embodiments, the oral product comprises propylene glycol.

Where included, the humectant is typically provided in an amount sufficient to provide desired moisture attributes to the composition. Further, in some instances, the humectant may impart desirable flow characteristics to the composition for depositing in a mold. It has also been found that the inclusion of a humectant, such as glycerine and/or propylene glycol, in the oral product may reduce the overall water activity of the oral product, and thus further improve the stability and shelf-life of the product.

When present in the oral product, the humectant (such as glycerine and/or propylene glycol) may be present in an amount of from about 0.01% to about 25% by weight of the oral product, such as from about 0.1% to about 20% by weight of the oral product, such as from about 0.5% to about 15% by weight of the oral product, such as from about 1% to about 10% by weight of the oral product, such as from about 5% to about 10% by weight of the oral product.

### Sweetener

In order to improve the sensory properties of the oral product according to the disclosure, one or more sweeteners may be added. The sweeteners can be any sweetener or combination of sweeteners, in natural or artificial form, or as a combination of natural and artificial sweeteners. Examples of natural sweeteners include fructose, sucrose, glucose, maltose, isomaltulose, mannose, galactose, lactose, stevia, honey, and the like. Examples of artificial sweeteners include sucralose, maltodextrin, saccharin, aspartame, acesulfame K, neotame and the like. In some embodiments, the sweetener comprises one or more sugar alcohols. Sugar alcohols are polyols derived from monosaccharides or disaccharides that have a partially or fully hydrogenated form. Sugar alcohols have, for example, about 4 to about 20 carbon atoms and include erythritol, arabitol, ribitol, isomalt, maltitol, dulcitol, iditol, mannitol, xylitol, lactitol, sorbitol, and combinations thereof (e.g., hydrogenated starch hydrolysates).

In some embodiments, the sweetener is selected from the group consisting of fructose, sucrose, glucose, maltose, mannose, galactose, lactose, stevia, honey, sucralose, isomaltulose, maltodextrin, saccharin, aspartame, acesulfame K, neotame, erythritol, arabitol, ribitol, isomalt, maltitol, dulcitol, iditol, mannitol, xylitol, lactitol, sorbitol, and mixtures thereof. In some embodiments, the sweetener is selected from the group consisting of sucralose, acesulfame K, aspartame, maltodextrin, mannitol, sucrose, and mixtures thereof. In some embodiments, the sweetener may be sucralose and/or acesulfame K.

When present in the oral product, the sweetener (such as sucralose and/or acesulfame K) may be present in an amount of from about 0.001% to about 5% by weight of the oral product, such as from about 0.01% to about 3% by weight of the oral product, such as from about 0.1% to about 1% by weight of the oral product.

### Binder

A binder (or combination of binders) may be employed in certain embodiments, in amounts sufficient to provide the desired physical attributes and physical integrity to the composition, and binders also often function as thickening or gelling agents. Typical binders can be organic or inorganic, or a combination thereof. Representative binders include cellulose derivatives (e.g., cellulose ethers), povidone, sodium alginate, starch-based binders, pectin, gums, carrageenan, pullulan, zein, and the like, and combinations thereof. In some embodiments, the binder comprises pectin or carrageenan or combinations thereof.

The amount of binder utilized in the composition can vary, but is typically up to about 30% by weight, and certain embodiments are characterized by a binder content of at least about 0.1% by weight, such as from about 1% to about 30% by weight, or about 1% to about 10% by weight, based on the total weight of the oral product.

In certain embodiments, the binder includes a gum, for example, a natural gum. As used herein, a natural gum refers to polysaccharide materials of natural origin that have binding properties, and which are also useful as a thickening or gelling agents. Representative natural gums derived from plants, which are typically water soluble to some degree, include xanthan gum, guar gum, gum arabic, ghatti gum, gum tragacanth, karaya gum, locust bean gum, gellan gum, and combinations thereof. When present, natural gum binder materials are typically present in an amount of up to about 5% by weight, for example, from about 0.1, about 0.2, about 0.3, about 0.4, about 0.5, about 0.6, about 0.7, about 0.8, about 0.9, or about 1%, to about 2, about 3, about 4, or about 5% by weight, based on the total weight of the oral product.

### Buffering Agent

In certain embodiments, the oral product of the present disclosure can comprise pH adjusters or buffering agents. Examples of pH adjusters and buffering agents that can be used include, but are not limited to, metal hydroxides (e.g., alkali metal hydroxides such as sodium hydroxide and potassium hydroxide), and other alkali metal buffers such as metal carbonates (e.g., potassium carbonate or sodium carbonate), or metal bicarbonates such as sodium bicarbonate, and the like. Where present, the buffering agent is typically present in an amount less than about 5% based on the weight of the oral product; for example, from about 0.5% to about 5%, such as, e.g., from about 0.75% to about 4%, from about 0.75% to about 3%, or from about 1% to about 2% by weight, based on the total weight of the oral product.

Non-limiting examples of suitable buffers include alkali metals acetates, glycinates, phosphates, glycerophosphates, citrates, carbonates, hydrogen carbonates, borates, or mixtures thereof. In some embodiments, the buffering agent is selected from the group consisting of sodium carbonate, sodium bicarbonate, sodium phosphate, ammonium phosphate, and mixtures thereof.

The oral product according to the disclosure may have any suitable pH. In certain embodiments, the oral product of the present disclosure has a pH of from about 4 to about 7. In certain embodiments, the oral product of the present disclosure has a pH of from about 4 to about 6.5. In certain embodiments, the oral product of the present disclosure has a pH of from about 4.5 to about 7. In certain embodiments, the oral product of the present disclosure has a pH of from about 4.5 to about 6.5. In certain embodiments, the oral product of the present disclosure has a pH of from about 4 to about 6.5. In certain embodiments, the oral product of the present disclosure has a pH of from about 4.5 to about 6. In certain embodiments, the oral product of the present disclosure has a pH of from about 5 to about 6.

The pH of the oral product may be measured by any suitable technique. For example, the pH of the oral product may be measured by contacting 5 grams of oral product with 95 g of water (100 g total) and then mixing for 5 minutes. After mixing the pH of the solution may be measured with a pH probe.

The emulsion according to the disclosure may have any suitable pH. In certain embodiments, the emulsion of the present disclosure has a pH of from about 4 to about 7. In certain embodiments, the emulsion of the present disclosure has a pH of from about 4.5 to about 7. In certain embodiments, the emulsion of the present disclosure has a pH of from about 5 to about 7. In certain embodiments, the emulsion of the present disclosure has a pH of from about 5.5 to about 7. In certain embodiments, the emulsion of the present disclosure has a pH of from about 6 to about 7. In certain embodiments, the emulsion of the present disclosure has a pH of from about 6 to about 6.5.

### Salt

In some embodiments, the oral product according to the disclosure comprises a salt (e.g., an alkali metal salt), typically employed in an amount sufficient to provide desired sensory attributes to the product. It has also been found that the inclusion of a salt, such as sodium chloride, in the oral product may reduce the overall water activity of the oral product, and thus further improve the stability and shelf-life of the product

Non-limiting examples of suitable salts include sodium chloride, potassium chloride, ammonium chloride, flour salt, sodium acetate, sodium citrate, and the like. When present, a representative amount of salt is at least about 0.5% by weight, such as at least about 1% by weight, such as at least about 1.5% by weight. In some embodiments, the oral product may comprise salt in an amount of from about 0.5% to about 10% by weight, such as from about 1% to about 7.5% by weight, such as from about 1.5% to about 5% by weight, based on the total weight of the oral product.

### Other Additives

Other additives can be included in the oral product. For example, the oral product can be processed, blended, formulated, combined, and/or mixed with other materials or ingredients. The additives can be artificial, or can be obtained or derived from herbal or biological sources. Examples of further types of additives include thickening or gelling agents (e.g., fish gelatin), preservatives (e.g., potassium sorbate, sodium benzoate, calcium propionate, and the like), disintegration aids, zinc or magnesium salts selected to be relatively water soluble for compositions with greater water solubility (e.g., magnesium or zinc gluconate) or selected to be relatively water insoluble for compositions with reduced water solubility (e.g., magnesium or zinc oxide), or combinations thereof. See, for example, those representative components, combination of components, relative amounts of those components, and manners and methods for employing those components, set forth in U.S. Pat. No. 9,237,769 to Mua et al., U.S. Pat. No. 7,861,728 to Holton, Jr. et al., US Pat. App. Pub. No. 2010/0291245 to Gao et al., and US Pat. App. Pub. No. 2007/0062549 to Holton, Jr. et al., each of which is incorporated herein by reference. Typical inclusion ranges for such additional additives can vary depending on the nature and function of the additive and the intended effect on the final composition, with an example range of up to about 10% by weight, (e.g., from about 0.1% to about 5% by weight) based on total weight of the oral product.

For example, where present, a preservative (such as potassium sorbate, sodium benzoate, calcium propionate, or the like) can be included in the oral product in an amount of from about 0.001% to about 5% by weight of the oral product, such as from about 0.01% to about 2.5% by weight of the oral product, such as from about 0.05% to about 1% by weight of the oral product. The inclusion of a preservative may serve to decrease the water activity of the oral product, thus further improving the stability and shelf-life of the oral product.

A colorant may be employed in amounts sufficient to provide the desired physical attributes to the oral product according to the present disclosure. Examples of colorants include various dyes and pigments, such as caramel coloring and titanium dioxide. The amount of colorant utilized in the oral product can vary, but when present is typically up to about 3% by weight, such as from about 0.1%, about 0.5%, or about 1%, to about 3% by weight, based on the total weight of the oral product.

The aforementioned additives can be employed together (e.g., as additive formulations) or separately (e.g., individual additive components can be added at different stages involved in the preparation of the final product). Furthermore, the aforementioned types of additives may be encapsulated as provided in the final product or composition. Exemplary encapsulated additives are described, for example, in WO2010/132444 to Atchley, which is incorporated by reference herein.

### Emulsion

In some embodiments, the oral product comprises an emulsion that comprises a continuous phase and a dispersed phase. In some embodiments, the emulsion comprises an oil phase and an aqueous phase. At least one active agent (such as a cannabinoid) may be included in the continuous phase and/or the dispersed phase. Where present, the emulsion may comprise an oil phase as the continuous phase or the dispersed phase. The emulsion may comprise an aqueous phase as the continuous phase or the dispersed phase. In some embodiments, the emulsion comprises an oil phase as the continuous phase and an aqueous phase as the dispersed phase (i.e. a water-in-oil emulsion). In some embodiments, the emulsion comprises an aqueous phase as the continuous phase and an oil phase as the dispersed phase (i.e. an oil-in-water emulsion). In some embodiments, the emulsion may be a water-in-oil-in-water emulsion. In some embodiments, the emulsion may be an oil-in-water-in-oil emulsion.

In some embodiments, the oral product comprises an emulsion in which at least one active agent (such as a cannabinoid) is included in the continuous and/or dispersed phase. In some embodiments, the oral product comprises an emulsion in which at least one cannabinoid (such as cannabidiol) is included in the continuous and/or dispersed phase.

Where present, the amount of the emulsion in the oral product may vary and may be any suitable amount for forming a product suitable for oral application. In some embodiments, the emulsion is present in the oral product in an amount of from about 1% to about 75% by weight of the oral product, such as from about 5% to about 60% by weight of the oral product, such as from about 10% to about 50% by weight of the oral product, such as from about 15% to about 45% by weight of the oral product, such as from about 20% to about 40% by weight of the oral product, such as from about 25% to about 40% by weight of the oral product, such as from about 30% to about 40% by weight of the oral product.

In some embodiments, the emulsion is present in the oral product in an amount of from about 20% to about 40% by weight of the oral product.

Where present, the emulsion may be in the form of a microemulsion. In some embodiments, the emulsion is in the form of a nanoemulsion. A nanoemulsion is a colloidal particulate system with particulates in the submicron size range. The particulates (referred to herein also as droplets or particles) are generally solid spheres, and the surfaces of such particulates are amorphous and lipophilic with a negative charge. Nanoemulsions generally comprise nanoscale particles or droplets having an average size of less than about 1,000 nm. Nanoemulsions as described herein comprise nanoparticles (or nanodroplets) of the dispersed phase emulsified in the continuous phase. In some embodiments, the nanoemulsion comprises nanoparticles of an oil phase emulsified in water or the aqueous phase.

Nanoemulsions as described herein generally comprise nanoscale particles having an average size of from about 10 nm to about 1,000 nm, for example, from about 10 nm to about 200 nm, from about 20 nm to about 100 nm, or from about 40 nm to about 100 nm. In some embodiments, the average particle size is about 100 nm, about 90 nm, about 80 nm, about 70 nm, about 60 nm, about 50 nm or about 40 nm. In some embodiments, the average particle size is from about 40 nm to about 60 nm. In some embodiments, the average particle size is from about 40 nm to about 60 nm, and the nanoemulsion is transparent.

The size of the nanoparticles may be determined by quasi-electric light scattering (QELS) as described in Bloomfield, Ann. Rev. Biophys. Bioeng., 10:421-450 (1981), incorporated herein by reference. It may also be measured by correlation spectroscopy that analyzes the fluctuation in scattering of light due to Brownian motion, or by transmission electron microscopy (TEM).

In embodiments in which the oral product comprises an emulsion, the oral product may further comprise one or more emulsifying agents. The one or more emulsifying agents may be contained within the emulsion. For example, the one or more emulsifying agents may be contained within the oil phase and/or the aqueous phase of an emulsion.

The emulsion (such as a nanoemulsion) in accordance with some embodiments may comprise one or more emulsifying agents. By “emulsifying agent” is meant a substance which aids in the formation and stabilization of emulsions by promoting dispersion of hydrophobic and hydrophilic (e.g., oil and water) components. In general, emulsifying agents are amphiphilic molecules chosen from, for example, nonionic and ionic amphiphilic molecules. The expression “amphiphilic molecule” means any molecule of bipolar structure comprising at least one hydrophobic portion and at least one hydrophilic portion and having the property of reducing the surface tension of water and of reducing the interface tension between water and an oily phase. Emulsifying agents/amphiphilic molecules as provided herein are also referred to as, for example, surfactants and emulsifiers.

The emulsifying agent may be included in the continuous phase, the dispersed phase, or in both the continuous phase and the dispersed phase of any emulsion. Alternatively or additionally, the emulsifying agent may be present at the interface of the dispersed and continuous phases.

In some embodiments, the emulsifying agent is selected from the group consisting of small molecule surfactants, phospholipids, proteins, polysaccharides, and mixtures thereof.

In some embodiments, the one or more emulsifying agents is selected from the group consisting of polyethylene glycol esters of fatty acids, propylene glycol esters of fatty acids, polysorbates, polyglycerol esters of fatty acids, polyglycerol polyricinoleate, sorbitan esters of fatty acid, sucrose esters of fatty acids, lecithins, enzyme treated lecithins, glycerin fatty acids esters, acetic acid esters of monoglycerides, lactic acid esters of monoglycerides, citric acid esters of monoglycerides, succinic acid esters of monoglycerides, diacetyl tartaric acid esters of monoglycerides, calcium stearoyl di lactate, chitin and chitosan derivatives, nature and modified starches, nature and modified hydrocolloids, nature and modified polysaccharides, nature and modified celluloses, nature and modified proteins, synthetic amphiphilic polymers, and mixtures thereof.

In some embodiments, the one or more emulsifying agents is selected from the group consisting of polyethylene glycol esters of fatty acids, propylene glycol esters of fatty acids, polysorbates, polyglycerol esters of fatty acids, polyglycerol polyricinoleate, sorbitan esters of fatty acid, sucrose esters of fatty acids, lecithins, glycerin fatty acids esters, acetic acid esters of monoglycerides, lactic acid esters of monoglycerides, citric acid esters of monoglycerides, succinic acid esters of monoglycerides, diacetyl tartaric acid esters of monoglycerides, calcium stearoyl di lactate, and mixtures thereof.

In some embodiments, the one or more emulsifying agents is selected from the group consisting of polyethylene glycol esters of fatty acids, polyethylene glycol esters of lecithin and mixtures thereof.

In some embodiments, the one or more emulsifying agents is selected from the group consisting of glycol distearate, sorbitan trioleate, sorbitan tristearate, sorbitan triisostearate, glyceryl isostearate, propylene glycol isostearate, glycol stearate, sorbitan sesquioleate, glyceryl stearate, lecithin, sorbitan oleate, sorbitan monostearate, sorbitan stearate, sorbitan isostearate, steareth-2, oleth-2, PEG-7 hydrogenated castor oil, laureth-2, sorbitan palmitate, laureth-3, glyceryl laurate, ceteth-2, PEG-30 dipolyhdroxystearate, glyceryl stearate SE, sorbitan stearate (and) sucrose cocoate, PEG-4 dilaurate, methyl glucose sesquistearate, PEG-8 dioleate, sorbitan laurate, PEG-40 sorbitan peroleate, laureth-4, PEG-7 glyceryl cocoate, PEG-20 almond glycerides, PEG-25 hydrogenated castor oil, stearamide MEA, glyceryl stearate (and) PEG-100 stearate, polysorbate 81, polysorbate 85, polysorbate 65, PEG-7 glyceryl cocoate, PEG-8 stearate, PEG-8 caprate, PEG-35 almond glycerides, PEG-6 laurate, laureth-7, steareth-10, isotrideceth-8, PEG-35 castor oil, isotrideceth-9, PEG-40 castor oil, ceteareth-12, laureth-9, PEG-40 hydrogenated castor oil, PEG-20 glyceryl isostearate, PEG-20 stearate, PEG-40 sorbitan perisostearate, PEG-7 olivate, cetearyl glucoside, PEG-8 oleate, polyglyceryl-3 methylglucose distearate, oleth-10, oleth-10/polyoxyl 10 oleyl ether NF, ceteth-10, PEG-8 laurate, cocamide MEA, polysorbate 60, polysorbate 80, isosteareth-20, PEG-60 almond glycerides, PEG-20 methyl glucose sesquistearate, ceteareth-20, oleth-20, steareth-20, steareth-21, ceteth-20, isoceth-20, polysorbate 20, polysorbate 40, ceteareth-25, ceteareth-30, PEG-30 stearate, laureth-23, PEG-75 lanolin, polysorbate 20, PEG-40 stearate, PEG-100 stearate, steareth-100, PEG-80 sorbitan laurate, polyoxyethylene stearate (e.g. polyoxyethylene (40) stearate), polyoxyethylene ether, and mixtures thereof.

In some embodiments, the one or more emulsifying agents have an overall HLB value in the range of from about 10 to about 15, such as from about 11 to about 15, such as from about 11 to about 14, such as from about 11 to about 13.5. As will be understood by one skilled in the art, HLB is the hydrophilic-lipophilic balance of an emulsifying agent or surfactant is a measure of the degree to which it is hydrophilic or lipophilic. The HLB value may be determined by calculating values for the different regions of the molecule, as described by Griffin in Griffin, William C. (1949), “Classification of Surface-Active Agents by ‘HLB’” (PDF), Journal of the Society of Cosmetic Chemists, 1 (5): 311-26 and Griffin, William C. (1954), “Calculation of HLB Values of Non-Ionic Surfactants” (PDF), Journal of the Society of Cosmetic Chemists, 5 (4): 249-56, and by Davies in Davies JT (1957), “A quantitative kinetic theory of emulsion type, I. Physical chemistry of the emulsifying agent” (PDF), Gas/Liquid and Liquid/Liquid Interface, Proceedings of the International Congress of Surface Activity, pp. 426-38. HLB value may be determined in accordance with the industry standard text book, namely “The HLB SYSTEM, a time-saving guide to emulsifier selection” ICI Americas Inc., Published 1976 and Revised, March, 1980. The HLB values of the emulsifiers described herein were determined in accordance with this standard method.

In some embodiments, the one or more emulsifying agents have an HLB value of from about 11 to about 15. In some embodiments, the one or more emulsifying agents have an HLB value of from about 11 to about 13.5. In some embodiments, the overall HLB value of the one or more emulsifying agents present in the oral product is from about 11 to about 15, such as from about 11 to about 13.5.

In some embodiments, the oral product comprises an emulsifying agent having an HLB value of from about 11 to about 15, wherein the emulsifying agent is selected from the group consisting of: stearamide MEA, glyceryl stearate (and) PEG-100 stearate, polysorbate 85, PEG-7 olivate, cetearyl glucoside, PEG-8 oleate, polyglyceryl-3 methylglucose distearate, oleth-10, oleth-10/polyoxyl 10 oleyl ether NF, ceteth-10, PEG-8 laurate, cocamide MEA, polysorbate 60, polysorbate 80, isosteareth-20, PEG-60 almond glycerides, PEG-20 methyl glucose sesquistearate, PEG-7 glyceryl cocoate, PEG-8 stearate, PEG-8 caprate, PEG-35 almond glycerides, PEG-6 laurate, laureth-7, steareth-10, isotrideceth-8, PEG-35 castor oil, isotrideceth-9, PEG-40 castor oil, ceteareth-12, laureth-9, PEG-40 hydrogenated castor oil, PEG-20 glyceryl isostearate, PEG-20 stearate, and mixtures thereof.

In some embodiments, the oral product comprises at least two emulsifying agents which have different HLB values. In some embodiments, the oral product comprises a first emulsifying agent with a low HLB value, and a second emulsifying agent with a high HLB value. In some embodiments, the oral product comprises a first emulsifying agent having an HLB value of from about 1 to about 9 (such as from about 2 to 9, such as from about 3 to 9, such as from about 3 to 8) and a second emulsifying agent having an HLB value of from about 10 to about 20 (such as from about 10 to 18, such as from about 11 to 17). In some embodiments, the overall (i.e., combined) HLB value of the first and second emulsifying agents is from about 11 to about 15, such as from about 11 to about 13.5.

The first emulsifying agent having an HLB value of from about 1 to about 9 may be selected from any suitable emulsifying agent having such an HLB value. For example, the first emulsifying agent may be an emulsifier having a HLB value of from about 1 to about 9 selected from mono and diglycerydes of fatty acid including glyceryl stearate and glyceryl oleate; fatty acid esters of C12-C22 fatty alcohols including fatty acid esters of cetyl alcohol and fatty acid esters of stearoyl alcohol, mixtures of fatty acid esters of cetyl alcohol and fatty acid esters of stearoyl alcohol, mixtures of fatty acid esters of cetyl alcohol and fatty acid esters of stearoyl alcohol wherein the fatty acids are derived from olive oil (such as cetearyl olivate), fatty acid esters of sorbitol including sorbitan oleate, fatty acid esters of sorbitol wherein the fatty acids are derived from olive oil (such as sorbitan olivate or cetearyl olivate), and mixtures thereof.

In some embodiments, the first emulsifying agent is an emulsifier having a HLB value of from about 1 to 9 selected from mono and diglycerydes of fatty acid, fatty acid esters of C12-C22 fatty alcohols, fatty acid esters of sorbitol, and mixtures thereof. In some embodiments, the first emulsifying agent is selected from the group consisting of glycol distearate, sorbitan trioleate, sorbitan tristearate, sorbitan triisostearate, glyceryl isostearate, propylene glycol isostearate, glycol stearate, sorbitan sesquioleate, glyceryl stearate, lecithin (such as soy lecithin), sorbitan oleate, sorbitan monostearate, sorbitan stearate, sorbitan isostearate, steareth-2, oleth-2, PEG-7 hydrogenated castor oil, laureth-2, sorbitan palmitate, laureth-3, glyceryl laurate, ceteth-2, PEG-30 dipolyhdroxystearate, glyceryl stearate SE, sorbitan stearate (and) sucrose cocoate, PEG-4 dilaurate, methyl glucose sesquistearate, PEG-8 dioleate, sorbitan laurate, PEG-40 sorbitan peroleate, and mixtures thereof.

In some embodiments, the first emulsifying agent is or comprises lecithin. In some embodiments, the first emulsifying agent is or comprises soy lecithin.

The second emulsifying agent may be selected from any suitable emulsifying agent having an HLB value of from about 10 to about 20. In some embodiments, the second emulsifying agent is an emulsifier having a HLB value of from 10 to 20 selected from fatty acid esters of polyethylene glycol, such as fatty acid esters of polyethylene glycol wherein the fatty acids are derived from coconut oil (including PEG 7), fatty acid esters of polyglycerol including fatty acid esters of polyglycerol and oleic acid (such as polyglyceryl 10 oleate), and mixtures thereof. In some embodiments, the second emulsifying agent is an emulsifier having a HLB value of from 10 to 20 selected from fatty acid esters of polyethylene glycol, fatty acid esters of polyglycerol, and mixtures thereof. In some embodiments, the second emulsifying agent may be selected from the group consisting of laureth-4, PEG-7 glyceryl cocoate, PEG-20 almond glycerides, PEG-25 hydrogenated castor oil, stearamide MEA, glyceryl stearate (and) PEG-100 stearate, polysorbate 81, polysorbate 85, polysorbate 65, PEG-7 glyceryl cocoate, PEG-8 stearate, PEG-8 caprate, PEG-35 almond glycerides, PEG-6 laurate, laureth-7, steareth-10, isotrideceth-8, PEG-35 castor oil, isotrideceth-9, PEG-40 castor oil, ceteareth-12, laureth-9, PEG-40 hydrogenated castor oil, PEG-20 glyceryl isostearate, PEG-20 stearate, PEG-40 sorbitan perisostearate, PEG-7 olivate, cetearyl glucoside, PEG-8 oleate, polyglyceryl-3 methylglucose distearate, oleth-10, oleth-10/polyoxyl 10 oleyl ether NF, ceteth-10, PEG-8 laurate, cocamide MEA, polysorbate 60, polysorbate 80, isosteareth-20, PEG-60 almond glycerides, PEG-20 methyl glucose sesquistearate, ceteareth-20, oleth-20, steareth-20, steareth-21, ceteth-20, isoceth-20, polysorbate 20, polysorbate 40, ceteareth-25, ceteareth-30, PEG-30 stearate, laureth-23, PEG-75 lanolin, polysorbate 20, PEG-40 stearate, PEG-100 stearate, steareth-100, PEG-80 sorbitan laurate, polyoxyethylene stearate (e.g. polyoxyethylene (40) stearate), polyoxyethylene ether, and mixtures thereof. In some embodiments, the second emulsifying agent is or comprises polyoxyethylene stearate (e.g. polyoxyethylene (40) stearate).

In some embodiments, the emulsifying agent is or comprises a combination of lecithin (e.g. soy lecithin) and polyoxyethylene stearate (e.g. polyoxyethylene (40) stearate).

In some embodiments, the one or more emulsifying agents comprises neutral, positively charged, or negatively charged natural or synthetic phospholipids molecules. Phospholipids are made up of two fatty acid tails and a phosphate group head, connected via a third molecule, glycerol. Non-limiting examples of natural phospholipids including lecithin (such as soy lecithin and/or egg lecithin), phosphatidyl choline-enriched lecithin, phosphatidyl serine-enriched lecithin, enzymatically modified lecithin, phosphatidylglycerol, phosphatidylinositol, phosphatidylethanolamine, phosphatidic acid, sphingomyelin, diphosphatidylglycerol, phosphatidylserine, phosphatidylcholine and cardiolipin; synthetic phospholipids including dimyristoylphosphatidylcholine, dimyristoylphosphatidylglycerol, di stearoylphosphatidylglycerol and dipalmitoylphosphatidylcholine; and hydrogenated or partially hydrogenated lecithins and phospholipids. Non-limiting examples of synthetic phospholipid derivatives include phosphatidic acid (DMPA, DPPA, DSPA), phosphatidylcholine (DDPC, DLPC, DMPC, DPPC, DSPC, DOPC, POPC, DEPC), phosphatidylglycerol (DMPG, DPPG, DSPG, POPG), phosphatidylethanolamine (DMPE, DPPE, DSPE DOPE), phosphatidylserine (DOPS), PEG phospholipid (mPEG-phospholipid, polyglycerin-phospholipid, functionalized-phospholipid, and terminal activated-phospholipid).

In some embodiments, the emulsifying agent comprises a surfactant, which may be ionic (anionic or cationic), zwitterionic or non-ionic, and which may be hydrophobic or hydrophilic. Examples of hydrophobic surfactants include, but are not limited to, Maisine 35-1, Imwitor 742, Capmul MCM, Capmul PG 12, Lauroglycol 90, Lauroglycol FCC, Caproyl 90, Captex 250, a fatty acid selected from the group consisting of octanoic acid, decanoic acid, undecanoic acid, lauric acid, myristic acid, palmitic acid, stearic acid, oleic acid, linoleic acid, and linolenic acid. As used herein, a hydrophobic surfactant may also be referred to as a poorly water soluble surfactant or a lipophilic surfactant.

Examples of hydrophilic surfactants may include, but are not limited to polyoxyethylene sorbitan fatty acid esters, hydrogenated castor oil ethoxylates, PEG mono- and di-esters of palmitic and stearic acids, fatty acid ethoxylates, and combinations thereof.

Examples of suitable surfactants generally include, but are not limited to: polyoxyethylene-sorbitan-fatty acid esters; e.g., mono- and tri-lauryl, palmityl, stearyl and oleyl esters; e.g., products of the type known as polysorbates and commercially available under the trade name Tween®; polyoxyethylene fatty acid esters, e.g., polyoxyethylene stearic acid esters of the type known and commercially available under the trade name Myrj®; polyoxyethylene ethers, such as those available under the trade name Brij®; polyoxyethylene castor oil derivatives, e.g., products of the type known and commercially available as Cremophors®. Particularly suitable are polyoxyl 35 castor oil (Cremophor® EL) and polyoxyl 40 hydrogenated castor oil (Cremophor® RH40); a-tocopherol, a-tocopheryl polyethylene glycol succinate (vitamin E TPGS), a-tocopherol palmitate and a-tocopherol acetate; PEG glyceryl fatty acid esters such as PEG-8 glyceryl caprylate/caprate (commercially known as Labrasol®), PEG-4 glyceryl caprylate/caprate (Labrafac Hydro WL 1219), PEG-32 glyceryl laurate (Gelucire 44/14), PEG-6 glyceryl mono oleate (Labrafil® M 1944 CS), PEG-6 glyceryl linoleate (Labrafil® M 2125 CS); propylene glycol mono- and di-fatty acid esters, such as propylene glycol laurate, propylene glycol caprylate/caprate; also diethyleneglycol-monoethylether (DGME), commercially known as Transcutol® (Gattefosse, Westwood, N.J.); sorbitan fatty acid esters, such as the type known and commercially available under the name Span® (e.g., Span 85); polyoxyethylene-polyoxypropylene co-polymers, e.g., products of the type known and commercially available as Pluronic® or Poloxamer®; glycerol triacetate; and monoglycerides and acetylated monoglycerides, e.g., glycerol monodicocoate (Imwitor® 928), glycerol monocaprylate (Imwitor® 308), and mono- and di-acetylated monoglycerides.

In some embodiments, the emulsifying agent is a surfactant, a phospholipid, an amphiphilic polysaccharide, an amphiphilic protein, or a combination thereof. In some embodiments, the one or more emulsifying agents is an ionic, zwitterionic, or non-ionic surfactant. In some embodiments, the one or more emulsifying agents comprises Tween 20, Tween 80, Span 20, Span 40, Span 60, Span 80, lecithin, Myrj 52, Brij 35, Brij 97, a hydrocolloid gum, a modified starch, or a combination thereof.

In some embodiments, the one or more emulsifying agents comprises a combination of lecithin and Myrj 52.

The concentration of the one or more emulsifying agents present in the disclosed emulsion may vary. The total concentration of the emulsifying agent may be in a range of up to about 30% by weight, for example from about 0.1% to about 25%, from about 5% to about 25%, or from about 10% to about 25% by weight based on the entirety of the emulsion. In some embodiments, the emulsion comprises a combination of lecithin and Myrj 52 in an amount of from about 0.1% to about 25%, from about 5% to about 25%, or from about 10% to about 25% by weight based on the entirety of the emulsion.

In some embodiments, the one or more emulsifying agents may be present in the emulsion in an amount of from about 0.1% to about 20% by weight of the oral product, such as from about 1% to about 15% by weight of the oral product, such as from about 2.5% to about 10% by weight of the oral product, such as from about 5% to about 10% by weight of the oral product. In some embodiments, the emulsion comprises a combination of lecithin and Myrj 52 in an amount of from about 0.1% to about 20% by weight of the oral product, such as from about 1% to about 15% by weight of the oral product, such as from about 2.5% to about 10% by weight of the oral product, such as from about 5% to about 10% by weight of the oral product.

In some embodiments, (a) providing an oral product comprises: (a) providing a filler and water, and optionally an active agent (such as a cannabinoid), and (b) contacting the filler and the water, and optionally the active agent to form an oral product.

In some embodiments, the active agent and/or the water may be provided in the form of an emulsion as described hereinabove.

In some embodiments, (a) further comprises contacting one or more additives with the filler and/or the water and/or the active agent. Such additives may be added before, during and/or after (b). In some embodiments, one or more additives is contacted with the filler and/or the water and/or the active agent before (b). In some embodiments, one or more additives is contacted with the filler and/or the water and/or the active agent during (b). In some embodiments, one or more additives is contacted with the filler and/or the water and/or the active agent after (b).

The manner by which the various components of the composition are combined may vary. As such, the overall composition with e.g., powdered composition components may be relatively uniform in nature. The components noted above, which may be in liquid or dry solid form, can be admixed in a pretreatment prior to mixture with any remaining components of the composition, or simply mixed together with all other liquid or dry ingredients. The various components of the composition may be contacted, combined, or mixed together using any mixing technique or equipment known in the art. Any mixing method that brings the composition ingredients into intimate contact can be used, such as a mixing apparatus featuring an impeller or other structure capable of agitation. Examples of mixing equipment include casing drums, conditioning cylinders or drums, liquid spray apparatus, conical-type blenders, ribbon blenders, mixers available as FKM130, FKM600, FKM1200, FKM2000 and FKM3000 from Littleford Day, Inc., Plough Share types of mixer cylinders, Hobart mixers, and the like. See also, for example, the types of methodologies set forth in U.S. Pat. No. 4,148,325 to Solomon et al.; U.S. Pat. No. 6,510,855 to Korte et al.; and U.S. Pat. No. 6,834,654 to Williams, each of which is incorporated herein by reference. In some embodiments, the components forming the composition are prepared such that the mixture thereof may be used in a molding process for forming the composition. Manners and methods for formulating compositions will be apparent to those skilled in the art. See, for example, the types of methodologies set forth in U.S. Pat. No. 4,148,325 to Solomon et al.; U.S. Pat. No. 6,510,855 to Korte et al.; and U.S. Pat. No. 6,834,654 to Williams, U.S. Pat. No. 4,725,440 to Ridgway et al., and U.S. Pat. No. 6,077,524 to Bolder et al., each of which is incorporated herein by reference.

In some embodiments, the method comprises mixing the filler, optionally at least one active agent (such as a cannabinoid), and a salt to form a first mixture; and adding water to the first mixture to form the oral product. In some embodiments, the method further comprises adding one or more binders to the first mixture. In some embodiments, the method further comprises adding a buffer, one or more sweeteners, a humectant, a flavoring agent, a taste modifying agent, or a combination thereof, to the first mixture. In some embodiments, the method further comprises adding additional water to the composition in order to provide the final oral product.

### Pouch

As described herein, (b) of the process comprises incorporating the oral product within the pouch to provide a pouched oral product.

As described herein, the pouch is saliva-permeable. This means that the pouch is made of a saliva-permeable pouch material. In some embodiments, the pouch material is a fleece material. In some embodiments, the pouch material is a non-woven material. In some embodiments, the pouch material is a non-woven fleece material. In some embodiments, the pouch material comprises viscose, such as viscose rayon fibers. In some embodiments, the pouch material comprises regenerated cellulose fibers. In some embodiments, the pouch material comprises polyester fibers; the polyester fibers may constitute the pouch material or may be included in combination with viscose (such as regenerated cellulose fibers).

In some embodiments, the pouch material comprises a binder that provides for heat sealing of the pouches during manufacture. In some embodiments, the pouch material comprises an acrylic binder. In some embodiments, the pouch material comprises an acrylic binder in combination with viscose and/or polyester fibers.

Suitable packets, pouches or containers of the type used for the manufacture of smokeless tobacco products are available under the tradenames CatchDry, Ettan, General, Granit, Goteborgs Rape, Grovsnus White, Metropol Kaktus, Mocca Anis, Mocca Mint, Mocca Wintergreen, Kicks, Probe, Prince, Skruf and TreAnkrare. The pouch provides a moisture-permeable container of a type that may be considered to be similar in character to the mesh-like type of material that is used for the construction of a tea bag. Components of the composition readily diffuse through the pouch and into the mouth of the user.

Non-limiting examples of suitable types of pouches are set forth in, for example, U.S. Pat. No. 5,167,244 to Kjerstad and U.S. Pat. No. 8,931,493 to Sebastian et al.; as well as US Patent App. Pub. Nos. 2016/0000140 to Sebastian et al.; 2016/0073689 to Sebastian et al.; 2016/0157515 to Chapman et al.; and 2016/0192703 to Sebastian et al., each of which is incorporated herein by reference. Pouches can be provided as individual pouches, or a plurality of pouches (e.g., 2, 4, 5, 10, 12, 15, 20, 25 or 30 pouches) can be connected or linked together (e.g., in an end-to-end manner) such that a single pouch or individual portion can be readily removed for use from a one-piece strand or matrix of pouches. The pouch may be formed of a moisture-permeable non-woven fabric, such as viscose for example.

An example pouch may be manufactured from materials, and in such a manner, such that during use by the user, the pouch undergoes a controlled dispersion or dissolution. Such pouch materials may have the form of a mesh, screen, perforated paper, permeable fabric, or the like. For example, pouch material manufactured from a mesh-like form of rice paper, or perforated rice paper, may dissolve in the mouth of the user. As a result, the pouch and composition each may undergo complete dispersion within the mouth of the user during normal conditions of use, and hence the pouch and composition both may be ingested by the user. Other examples of pouch materials may be manufactured using water dispersible film forming materials (e.g., binding agents such as alginates, carboxymethylcellulose, xanthan gum, pullulan, and the like), as well as those materials in combination with materials such as ground cellulosics (e.g., fine particle size wood pulp). Preferred pouch materials, though water dispersible or dissolvable, may be designed and manufactured such that under conditions of normal use, a significant amount of the composition contents permeate through the pouch material prior to the time that the pouch undergoes loss of its physical integrity. If desired, flavoring ingredients, disintegration aids, and other desired components, may be incorporated within, or applied to, the pouch material.

As described herein, in (b), the oral product is incorporated into the pouch to provide a pouched oral product. The oral product is thus contained in pouches. The pouched oral products may be packaged in a manner and using the types of components used for the manufacture of conventional snus types of products.

The amount of the oral product incorporated into each pouched product unit, for example, a pouch, may vary. In some embodiments, the weight of the oral product within each pouch is at least about 50 mg, for example, from about 50 mg to about 2 grams, from about 100 mg to about 1.5 grams, or from about 200 to about 700 mg. In some embodiments, the weight of the oral product within each pouch may be from about 100 mg to about 300 mg. For a larger embodiment, the weight of the material within each pouch may be from about 300 mg to about 700 mg. If desired, other components can be contained within each pouch. For example, at least one flavored strip, piece or sheet of flavored water dispersible or water soluble material (e.g., a breath-freshening edible film type of material) may be disposed within each pouch along with or without at least one capsule. Such strips or sheets may be folded or crumpled in order to be readily incorporated within the pouch. See, for example, the types of materials and technologies set forth in U.S. Pat. No. 6,887,307 to Scott et al. and U.S. Pat. No. 6,923,981 to Leung et al.; and The EFSA Journal (2004) 85, 1-32; which are incorporated herein by reference.

In some embodiments, (b) incorporating the oral product within the pouch comprises inserting the oral product into an open-ended pouch; i.e. in the form of a tube or pocket in which one end of the pouch material has been sealed, whilst the other end remains open. Once the oral product has been inserted into the tube of the pouch material, the open end is closed via sealing. Sealing may be carried out via heat welding and/or ultrasonic welding. The pouch material may subsequently be cut in order to sever the sealed pouch from any excess material.

### Application of Cannabinoid

According to some embodiments described herein, the process comprises (c) applying a cannabinoid to the surface of the pouch or applying a cannabinoid to the oral product through the pouch.

In some embodiments, (c) comprises applying a cannabinoid to the surface of the pouch. Such embodiment typically comprises applying the cannabinoid to the external surface of the pouch or pouch material. As described herein, the “external surface” or “outer surface” of the pouch is the surface of the pouch that is not in contact with the oral product incorporated therein. In some embodiments, the cannabinoid may be applied to the surface of the pouch such that at least part of the surface contains a cannabinoid dispersed thereon. In some embodiments, the cannabinoid may be applied such that the entirety of the outer surface has a cannabinoid dispersed thereon. The cannabinoid may be dispersed evenly across the outer surface, or may be distributed such that one or more discrete sections of the outer surface of the pouch have a cannabinoid dispersed thereon.

In some embodiments, the cannabinoid is applied to the surface of the pouch (such as external surface of the pouch) via spraying, dropping, spreading, embossing, coating and/or dispersing the cannabinoid onto the pouch. In some embodiments, the cannabinoid is applied to the surface of the pouch (such as external surface of the pouch) via spraying, dropping or spreading the cannabinoid onto the pouch. In some embodiments, the cannabinoid is applied to the surface of the pouch through at least one spray nozzle or atomizer. The spray nozzle may be coupled to a liquid reservoir configured to introduce liquid cannabinoid “mist” onto the pouch material. The cannabinoid may be applied to the surface of the pouch in intermittent pulses. Alternatively, the cannabinoid may be applied to the surface of the pouch as a constant flow.

In some embodiments, the cannabinoid is applied to the surface of the pouch (such as external surface of the pouch) via spraying using an apparatus comprising a spray nozzle coupled to a source of liquid cannabinoid via a pump. The spray nozzle is configured to spray a mist of the liquid cannabinoid directly onto the pouch material. In practice, the cannabinoid may be sprayed onto the pouch material in an in-line process in which multiple pouched oral products are produced, and therefore multiple pouches are being sprayed with the cannabinoid. The spray system may be configured to spray a pulse of liquid cannabinoid mist onto the pouch material at regular intervals during the process. A controller may be connected to the apparatus, and may control the system to coordinate spraying pulses of cannabinoid onto the pouch material when each individual pouch is being processed. This may provide the correct dose and even distribution of cannabinoid per pouch or per package (or container full of pouches), and/or to stop spraying the cannabinoid between pouch changeover. However, it is also envisaged that the system may provide a continuous spray of cannabinoid over a period of time such that multiple pouches may be sprayed using a continuous spray. Again, this may be controlled by a controller to control the system to coordinate continuous spraying of cannabinoid onto pouch material(s), and to provide the correct dose of cannabinoid per pouch. Alternatively or additionally, the pouch may be immersed in a substance comprising the cannabinoid. In other words, the cannabinoid may be applied to the surface (such as external surface) of the pouch by immersing the pouch in the cannabinoid. For example, the pouch may be immersed directly into the cannabinoid or into an oil-based mixture that comprises the cannabinoid dissolved therein. In some embodiments, (c) may comprise immersing the pouch in a substance comprising the cannabinoid for a period of from about 0.1 minutes to about 10 minutes, such as from about 0.5 minutes to about 5 minutes, such as from about 1 minute to about 2 minutes.

In certain embodiments, one or more active ingredients as described herein are included in the oral product within the pouch, and a cannabinoid is further disposed in or on the external surface of the pouched product (e.g., on or in the pouch material as disclosed herein). In some embodiments, separate location of the active ingredients may allow differential release profiles (e.g., one active ingredient may be rapidly available to the mouth and/or digestive system, and the other active ingredient may be released more gradually with product use). For example, in some embodiments, the composition (or oral product) within the pouched product may include at least one cannabinoid, and at least one cannabinoid may also be disposed in or on the external surface of the pouched product (e.g., on or in the pouch material as disclosed herein). Alternatively or in addition, at least one further and distinct active agent may be included in the oral product within the pouch, and at least one cannabinoid may be included in or on the external surface of the pouch.

It has been found that application of the cannabinoid to the surface of the pouch or pouch material may provide an advantageous release profile of the cannabinoid. For example, in embodiments in which the cannabinoid is only present in the pouched oral product on the pouch material itself (i.e., the oral product does not contain any cannabinoid), the presence of the cannabinoid on the pouch material may lead to relatively rapid release of the cannabinoid when placed in the oral cavity. This is because the cannabinoid may not be tightly bound the surface of the pouch, and it also does not need to diffuse out through the pouch material from the oral product contained therein. This may allow for a faster “hit” of cannabinoid to be delivered to the user. Due to the rapid release, the cannabinoid may also be absorbed into the bloodstream of the user more rapidly.

In some embodiments, the cannabinoid is applied to the oral product through the pouch or pouch material. In some embodiments, the cannabinoid is applied to the oral product through the pouch via injection.

Alternatively or additionally, the cannabinoid is applied to the oral product through the pouch via spraying, dropping or spreading the cannabinoid onto the pouch and/or via immersing the pouch in a substance comprising the cannabinoid.

In the process described hereinabove, the cannabinoid is applied to the pouch or to the oral product through the pouch after incorporation of the oral product within the pouch. In some embodiments, the above-described methods of applying the cannabinoid to the oral product through the pouch may result in some cannabinoid being applied both to the pouch material itself and also to the oral product within the pouch. Therefore, in some embodiments, (c) comprises applying the cannabinoid to both the oral product contained within the pouch and to the pouch material itself.

The inventors have found that application of the cannabinoid to the oral product after incorporation of the oral product within the pouch may lead to improved freshness of the cannabinoid as it may not degrade or leach out of the product during storage. For example, the cannabinoid may advantageously be applied to the oral product by the user immediately prior to the use of the pouched oral product, thereby imparting freshness to the product. Furthermore, application of the cannabinoid to the oral product through the pouch may inevitably lead to some cannabinoid being deposited or applied to the external surface of the pouch material itself. As such, the pouched oral product may provide desirable release characteristics due to the separate location of the active ingredients; this separate location may allow differential release profiles (e.g., the portion of the cannabinoid disposed on the surface of the pouch may be rapidly available to the mouth and/or digestive system, and the other portion of the cannabinoid incorporated in the oral product within the pouch may be released more gradually with product use).

According to some further broad aspects described herein, the process comprises applying the cannabinoid to the surface of the pouch (or pouch material) prior to incorporation of the oral product into the pouch. In such embodiments, the cannabinoid may be applied to the surface of the pouch in the same ways as those described hereinabove.

According to some embodiments described herein, there is also provided a process for preparing a pouched oral product, wherein the pouched oral product comprises a saliva permeable pouch and an oral product incorporated within the pouch, the process comprising: (a) providing the oral product, and (b) incorporating the oral product within the pouch to provide a pouched oral product; wherein the pouch material contains a cannabinoid or has a cannabinoid on its outer surface.

The cannabinoid may be applied to the pouch material (such that the pouch material contains a cannabinoid or has a cannabinoid on its outer surface) according to any one of the processes described hereinabove. The cannabinoid may be applied to the pouch material after (b) incorporating the oral product within the pouch. Alternatively or in addition, the cannabinoid may be applied to the pouch material before the oral product is incorporated within the pouch. Therefore, in some embodiments, the pouch material may comprise the cannabinoid without any contamination of the oral product with the cannabinoid during processing. As such, in some embodiments, the cannabinoid is applied to the pouch material, but not to the oral product within the pouch.

Therefore, in some embodiments, the process comprises:
(a) providing the oral product,
(b) applying a cannabinoid to the surface of the pouch, and subsequently
(c) incorporating the oral product within the pouch to provide a pouched oral product;
   such that the pouch material contains a cannabinoid or has a cannabinoid on its outer surface.

Alternatively, in some embodiments, the process comprises:
(a) providing the oral product,
(b) incorporating the oral product within the pouch to provide a pouched oral product; and subsequently
(c) applying a cannabinoid to the surface of the pouch,
   such that the pouch material contains a cannabinoid or has a cannabinoid on its outer surface.

The cannabinoid applied to the surface of the pouch or through the pouch may be a cannabinoid as described hereinabove. In some embodiments, the cannabinoid is or comprises cannabidiol. In some embodiments, the cannabinoid is cannabidiol.

In some embodiments, the cannabinoid is applied to the surface of the pouch in any suitable amount to provide the desired physiological effect on the user upon insertion to the oral cavity. In some embodiments, the cannabinoid is applied to the surface of the pouch such that the pouch material comprises cannabinoid in an amount of from about 0.001% to about 10% by weight of the total pouched oral product, such as in an amount of from about 0.01% to about 5% by weight of the total pouched oral product, such as in an amount of from about 0.1% to about 1% by weight of the total pouched oral product. In some embodiments, the cannabinoid is applied to the surface of the pouch such that the pouch material comprises cannabinoid in an amount of from about 0.01% to about 30% by weight of the pouch material, such as in an amount of from about 0.1% to about 20% by weight of the pouch material, such as from about 0.5% to about 15% by weight of the pouch material, such as in an amount of from about 1% to about 10% by weight of the pouch material.

### Pouched Oral Product

In accordance with some embodiments described herein, there is provided a pouched oral product, wherein the pouched oral product comprises a saliva permeable pouch and an oral product incorporated within the pouch, wherein the pouch material contains a cannabinoid or has a cannabinoid on its outer surface.

The cannabinoid may be applied to the pouch material via a process as described hereinabove. In some embodiments, the cannabinoid is applied to the pouch material via spraying, dropping, or spreading the cannabinoid onto the pouch, or by immersing the pouch in a substance comprising the cannabinoid. In some embodiments, the pouch material contains a cannabinoid or has a cannabinoid on its outer surface in an amount of from about 0.001% to about 10% by weight of the total pouched oral product, such as in an amount of from about 0.01% to about 5% by weight of the total pouched oral product, such as in an amount of from about 0.1% to about 1% by weight of the total pouched oral product. In some embodiments, the pouch material contains a cannabinoid or has a cannabinoid on its outer surface in an amount of from about 0.01% to about 30% by weight of the pouch material, such as in an amount of from about 0.1% to about 20% by weight of the pouch material, such as from about 0.5% to about 15% by weight of the pouch material, such as in an amount of from about 1% to about 10% by weight of the pouch material.

In accordance with some embodiments described herein, there is provided a pouched oral product obtained or obtainable by a process as defined herein. In some embodiments, the pouched oral product is obtained by a process as defined herein.

The pouched oral product may comprise each of the features described hereinabove.

In some embodiments, the product of the present disclosure is in the form of a pouched oral product. Such a pouched product comprises the oral product disposed within a moisture-permeable container (e.g., a water-permeable pouch or saliva-permeable pouch). For example, the pouched product may comprise the oral product in a powder form incorporated within the saliva-permeable pouch. Therefore, according to some embodiments described herein, there is provided a pouched oral product comprising a saliva permeable pouch and an oral product incorporated within the pouch, wherein the oral product is in powder form.

Such compositions in the moisture-permeable pouch format are typically used by placing one pouch containing the composition in the mouth of a human subject/user. Generally, the pouch is placed somewhere in the oral cavity of the user, for example under the lips, in the same way as moist snuff products are generally used. The pouch preferably is not chewed or swallowed. Exposure to saliva then causes some of the components of the composition therein (e.g., flavoring agents and/or active ingredients) to pass through e.g., the moisture-permeable pouch and provide the user with flavor and satisfaction, and the user is not required to spit out any portion of the composition. After about 10 minutes to about 60 minutes, typically about 15 minutes to about 45 minutes, of use/enjoyment, substantial amounts of the composition have been ingested by the human subject, and the pouch may be removed from the mouth of the human subject for disposal.

In accordance with some embodiments described herein, there is provided a package containing at least one pouched oral product as defined herein.

A pouched product as described herein can be packaged within any suitable inner packaging material and/or outer container. See also, for example, the various types of containers for smokeless types of products that are set forth in U.S. Pat. No. 7,014,039 to Henson et al.; U.S. Pat. No. 7,537,110 to Kutsch et al.; U.S. Pat. No. 7,584,843 to Kutsch et al.; U.S. Pat. No. 8,397,945 to Gelardi et al., U.S. Pat. No. D592,956 to Thiellier; U.S. Pat. No. D594,154 to Patel et al.; and U.S. Pat. No. D625,178 to Bailey et al.; US Pat. Pub. Nos. 2008/0173317 to Robinson et al.; 2009/0014343 to Clark et al.; 2009/0014450 to Bjorkholm; 2009/0250360 to Bellamah et al.; 2009/0266837 to Gelardi et al.; 2009/0223989 to Gelardi; 2009/0230003 to Thiellier; 2010/0084424 to Gelardi; and 2010/0133140 to Bailey et al; 2010/0264157 to Bailey et al.; and 2011/0168712 to Bailey et al. which are incorporated herein by reference. For example, the package may be a tin or plastic container which contains one or a plurality of the pouched oral products.

### EXAMPLES

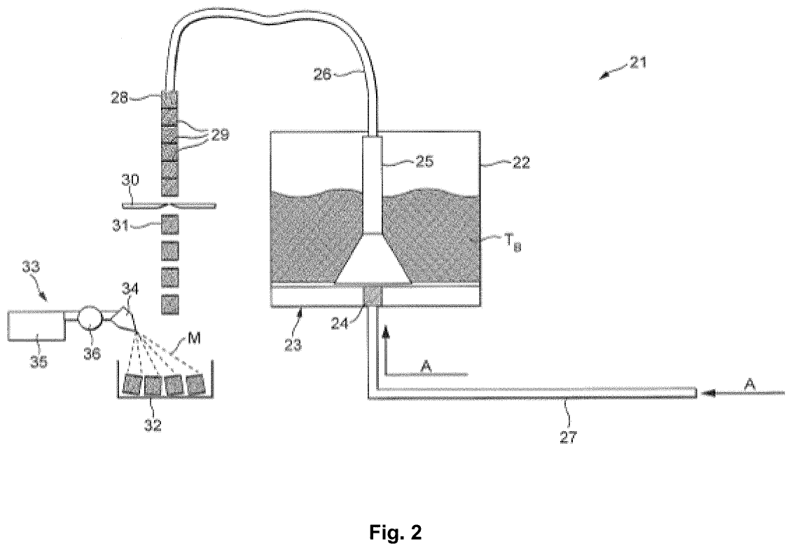

An apparatus 21 for producing oral product pouches according to the present invention is shown schematically in FIG. 2 and comprises hopper 22 to hold loose oral product TB, a plug-forming means 23 at the bottom of the hopper 22 to form the loose oral product into individual metered plugs 24 of oral product, and a guide duct 25 for the formed plugs of oral product 24 to travel through to a dosing pipe 26 connected to the other end of the guide duct 25 and on to a sleeve of pouch material 28 which is then sealed closed between each plug with a weld seam 29 and cut at each seam with a cutter 30 to form individual oral product pouch portions 31. These individual oral product pouches 31 are then packed into containers 32. A pipe 27 is connected to the base of the hopper 22 and is connected to a source of compressed air (not shown) to provide a compressed air flow (shown by arrows ‘A’) though the pipe 27, into the guide duct 25 to propel each plug of oral product 24 though the guide duct 25, through the dosing pipe 26 and into the pouch material sleeve 28.

The apparatus 21 includes an additive system 33 located proximate the end of the process line where the individual oral product pouches 31 are packed into the container 32. The additive system 33 comprises a spray nozzle 34 coupled to a source of cannabinoid via a pump 36, the nozzle 34 being configured to spray a mist M of cannabinoid additive directly onto the oral product pouches as the individual pouches 31 are delivered thereto. The container 32 is to be sealed and eventually sold to consumers.

In use, the pouches 31 are formed in the conventional manner described above. As the pouches are delivered into the container 32, the additive system 33 sprays the cannabinoid directly onto the oral product pouches.

The additive system 33 may be configured to spray a pulse of cannabinoid mist M onto the oral product pouches at regular intervals during filling of the container 32 with pouches 31. A controller (not shown) may be connected to the pouch-forming apparatus and may control the additive system 33 to co-ordinate spraying pulses of cannabinoid additive M onto the oral product pouches when each individual container 32 is being filled, and to provide the correct dose and even distribution of additive per pouch, and/or to stop spraying the additive between container change-over when one container is full and the next empty container takes its place. However, it is also envisaged that the additive system 33 may provide a continuous spray of cannabinoid additive M onto the oral product pouches. Again, this could be controlled by a controller (not shown) to control the additive system 33 to coordinate continuous spraying of cannabinoid additive M onto the oral product pouches when each individual container 32 is being filled, and to provide the correct dose of additive per pouch, and/or stop spraying the additive between container change-over when one container is full and the next empty container takes its place. Alternatively, the additive system 33 may simply provide a continuous spray of cannabinoid additive M onto the oral product pouches for the duration of time the processing system is in operation, and container 32 change-over may be quick to minimise cannabinoid additive wastage. A system comprising a controller would make most efficient use of the cannabinoid additive, avoiding any wastage, whereas the latter system without a controller could be simpler and therefore less expensive in terms of apparatus costs.

The various embodiments described herein are presented only to assist in understanding and teaching the claimed features. These embodiments are provided as a representative sample of embodiments only, and are not exhaustive and/or exclusive. It is to be understood that advantages, embodiments, examples, functions, features, structures, and/or other aspects described herein are not to be considered limitations on the scope of the invention as defined by the claims or limitations on equivalents to the claims, and that other embodiments may be utilised and modifications may be made without departing from the scope of the claimed invention. Various embodiments of the invention may suitably comprise, consist of, or consist essentially of, appropriate combinations of the disclosed elements, components, features, parts, steps, means, etc., other than those specifically described herein. In addition, this disclosure may include other inventions not presently claimed, but which may be claimed in future.

1. A process for preparing a pouched oral product, wherein the pouched oral product comprises a saliva permeable pouch and an oral product incorporated within the pouch, the process comprising:
(a) providing the oral product,
(b) incorporating the oral product within the pouch to provide a pouched oral product; and
(c) applying a cannabinoid to the surface of the pouch or applying a cannabinoid to the oral product through the pouch.

2. The process of claim 1, wherein (c) comprises applying a cannabinoid to the surface of the pouch.

3. The process of claim 2, wherein (c) comprises applying the cannabinoid to the pouch via spraying, dropping or spreading the cannabinoid onto the pouch, or via immersing the pouch in a substance comprising the cannabinoid.

4. The process of claim 1, wherein (c) comprises applying the cannabinoid to the oral product through the pouch.

5. The process of claim 4, wherein (c) comprises applying the cannabinoid to the oral product through the pouch via injection.

6. The process of claim 1, wherein the pouch comprises a nonwoven fleece material.

7. The process of claim 6, wherein the pouch comprises viscose.

8. The process of claim 1, wherein the cannabinoid is selected from the group consisting of cannabigerol (CBG), cannabichromene (CBC), cannabidiol (CBD), tetrahydrocannabinol (THC), cannabinol (CBN) and cannabinodiol (CBDL), cannabicyclol (CBL), cannabivarin (CBV), tetrahydrocannabivarin (THCV), cannabidivarin (CBDV), cannabichromevarin (CBCV), cannabigerovarin (CBGV), cannabigerol monomethyl ether (CBGM), cannabinerolic acid, cannabidiolic acid (CBDA), Cannabinol propyl variant (CBNV), cannabitriol (CBO), tetrahydrocannabmolic acid (THCA), tetrahydrocannabivarinic acid (THCV A), and mixtures thereof.

9. The process of claim 1, wherein the cannabinoid comprises cannabidiol.

10. The process of claim 9, wherein the cannabinoid comprises cannabidiol in an amount of at least 98% by weight of the cannabinoid.

11. The process of claim 1, wherein the oral product provided in (a) does not contain any cannabinoid.

12. The process of claim 1, wherein the oral product provided in (a) comprises a cannabinoid.

13. The process of claim 12, wherein the oral product provided in (a) comprises a cannabinoid contained in an emulsion that comprises a dispersed phase and a continuous phase.

14. The process of claim 1, wherein the oral product provided in (a) comprises a filler.

15. The process of claim 14, wherein the filler is a cellulose material selected from the group consisting of maize fiber, oat fiber, barley fiber, rye fiber, buckwheat fiber, sugar beet fiber, bran fiber, bamboo fiber, wood pulp fiber, cotton fiber, citrus pulp fiber, grass fiber, willow fiber, poplar fiber, cocoa fiber, derivatives thereof, and combinations thereof.

16. A process for preparing a pouched oral product, wherein the pouched oral product comprises a saliva permeable pouch and an oral product incorporated within the pouch, the process comprising:
(a) providing the oral product; and
(b) incorporating the oral product within the pouch to provide a pouched oral product, wherein the pouch material contains a cannabinoid or has a cannabinoid on its outer surface.

17. The process of claim 16, wherein the cannabinoid is applied to the pouch via spraying, dropping, or spreading the cannabinoid onto the pouch, or via immersing the pouch in a substance comprising the cannabinoid.

18. A pouched oral product, wherein the pouched oral product comprises a saliva permeable pouch and an oral product incorporated within the pouch, wherein the pouch material contains a cannabinoid or has a cannabinoid on its outer surface.

19. The pouched oral product of claim 18, wherein the oral product incorporated within the pouch does not comprise any cannabinoid.

20. The pouched oral product of claim 18, wherein the oral product incorporated within the pouch further comprises a cannabinoid.

21. The pouched oral product of claim 18, wherein the total amount of cannabinoid in the pouched oral product is from about 0.5% to about 20% of the pouched oral product.

22. A pouched oral product obtained or obtainable by the process of claim 1.

23. A package containing at least one pouched oral product as defined in claim 18.
